SEQUENCE LISTING (15 sequences)

SEQ ID NO: 1 - protein, 8 aa, synthetic construct
GYTFTSYW

SEQ ID NO: 2 - protein, 8 aa, synthetic construct
IYPGSGST

SEQ ID NO: 3 - protein, 14 aa, synthetic construct
TMRYGGYYGYYFDY

SEQ ID NO: 4 - protein, 7 aa, synthetic construct
SSISSNY

SEQ ID NO: 5 - protein, 9 aa, synthetic construct
QQGSSIPLT

SEQ ID NO: 6 - protein, 121 aa, synthetic construct
QVQLQQPGSELVRPGASVKLSCKASGYTFTSYWMHWVKQRHGQGLEWIGNIYPGSGSTNYDEKFKSKGTLTVDTSSSTAYMHLGSLTSEDSAVYYCTMRYGGYYGYYFDYWGQGTTLTVSS

SEQ ID NO: 7 - protein, 451 aa, synthetic construct
QVQLQQPGSELVRPGASVKLSCKASGYTFTSYWMHWVKQRHGQGLEWIGNIYPGSGSTNYDEKFKSKGTLTVDTSSSTAYMHLGSLTSEDSAVYYCTMRYGGYYGYYFDYWGQGTTLTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 109 aa, synthetic construct
EIVLTQSPTTMAASPGEKITITCSTTSSISSNYLHWYQQKPGFSPKLLIYRTSNLASGVPARFSGSGSGTSYSLTIGTMEAEDVATYYCQQGSSIPLTFGAGTKLELKR

SEQ ID NO: 9 - protein, 215 aa, synthetic construct
EIVLTQSPTTMAASPGEKITITCSTTSSISSNYLHWYQQKPGFSPKLLIYRTSNLASGVPARFSGSGSGTSYSLTIGTMEAEDVATYYCQQGSSIPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 10 - DNA, 363 bp, synthetic construct
  source 1..363: mol_type = other DNA
caggtccaactgcagcaacctgggtctgagctggtgaggcctggagcttcagtgaagctgtcctgcaaggcttctggctacacattcaccagctactggatgcactgggtgaagcagaggcatggacaaggccttgagtggattggaaatatttatcctggtagtggtagtactaactacgatgagaagttcaagagcaagggcacactgactgtagacacatcctccagcacagcctacatgcacctcggcagcctgacatctgaggactctgcggtctattactgtacaatgcgctacggtggttactacgggtactactttgactactggggccaaggcaccactctcacagtctcctca

SEQ ID NO: 11 - DNA, 327 bp, synthetic construct
  source 1..327: mol_type = other DNA
gaaattgtgctcacccagtctccaaccaccatggctgcatctcccggggagaagatcactatcacctgcagtaccacctcaagtataagttccaattacttgcattggtatcagcagaagccaggattctcccctaaactcttgatttataggacatccaatctggcttctggagtcccagctcgcttcagtggcagtgggtctgggacctcttactctctcacaattggcaccatggaggctgaagatgttgccacttactactgccagcagggtagtagtataccgctcacgttcggtgctgggaccaagctggagctgaaacgg

SEQ ID NO: 12 - protein, 726 aa, synthetic construct
MVPCTLLLLLAAALAPTQTRAQVQLQQPGSELVRPGASVKLSCKASGYTFTSYWMHWVKQRHGQGLEWIGNIYPGSGSTNYDEKFKSKGTLTVDTSSSTAYMHLGSLTSEDSAVYYCTMRYGGYYGYYFDYWGQGTTLTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKEGRGSLLTCGDVEENPGPMVPCTLLLLLAAALAPTQTRAEIVLTQSPTTMAASPGEKITITCSTTSSISSNYLHWYQQKPGFSPKLLIYRTSNLASGVPARFSGSGSGTSYSLTIGTMEAEDVATYYCQQGSSIPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 13 - DNA, 2178 bp, synthetic construct
  source 1..2178: mol_type = other DNA
atggtaccgtgcacgctgctcctgctgttggcggccgccctggctccgactcagacccgcgcgcaggtccaactgcagcaacctgggtctgagctggtgaggcctggagcttcagtgaagctgtcctgcaaggcttctggctacacattcaccagctactggatgcactgggtgaagcagaggcatggacaaggccttgagtggattggaaatatttatcctggtagtggtagtactaactacgatgagaagttcaagagcaagggcacactgactgtagacacatcctccagcacagcctacatgcacctcggcagcctgacatctgaggactctgcggtctattactgtacaatgcgctacggtggttactacgggtactactttgactactggggccaaggcaccactctcacagtctcctcagccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctgaactcctggggggaccgtcagtcttcctcttccccccaaaacccaaggacaccctcatgatctcccggacccctgaggtcacatgcgtggtggtggcagtgagccacgaagaccctgaggtcaagttcaactggtacgtggacggcgtggaggtgcataatgccaagacaaagccgcgggaggagcagtacgcaagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagagggcagaggcagcctgctgacctgcggcgacgtggaggagaaccccggccccatggtaccgtgcacgctgctcctgctgttggcggccgccctggctccgactcagacccgcgcggaaattgtgctcacccagtctccaaccaccatggctgcatctcccggggagaagatcactatcacctgcagtaccacctcaagtataagttccaattacttgcattggtatcagcagaagccaggattctcccctaaactcttgatttataggacatccaatctggcttctggagtcccagctcgcttcagtggcagtgggtctgggacctcttactctctcacaattggcaccatggaggctgaagatgttgccacttactactgccagcagggtagtagtataccgctcacgttcggtgctgggaccaagctggagctgaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 14 - protein, 21 aa, synthetic construct
MVPCTLLLLLAAALAPTQTRA

SEQ ID NO: 15 - protein, 451 aa, synthetic construct
QVQLQQPGSELVRPGASVKLSCKASGYTFTSYWMHWVKQRHGQGLEWIGNIYPGSGSTNYDEKFKSKGTLTVDTSSSTAYMHLGSLTSEDSAVYYCTMRYGGYYGYYFDYWGQGTTLTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2026/0028405 A1
(43) Pub. Date: Jan. 29, 2026

(54) ANTI-CD16A ANTIBODIES AND METHODS OF USE THEREOF

(71) Applicant: Icahn School of Medicine at Mount Sinai, New York, NY (US)

(72) Inventors: Lucas Ferrari De Andrade, New York, NY (US); Bruna Taciane Da Silva Bortoleti, New York, NY (US)

(21) Appl. No.: 18/994,323

(22) PCT Filed: Jul. 26, 2023
(86) PCT No.: PCT/US23/71066

§ 371 Date: Jan. 14, 2025

Related U.S. Application Data
(60) Provisional application No. 63/369,720, filed on Jul. 28, 2022.

(51) Int. Cl. C07K 16/28; A61K 39/00; A61K 39/395; A61P 35/00; C12N 15/11; C12N 15/79

(52) U.S. Cl. CPC C07K 16/283; A61K 39/39533; A61P 35/00; C07K 16/2863; C12N 15/11; C12N 15/79; A61K 2039/507; A61K 2039/585; C07K 2317/24; C07K 2317/31; C07K 2317/40; C07K 2317/52; C07K 2317/565

(57) ABSTRACT

Provided herein are recombinant antibodies and antigen-binding fragments thereof useful for binding to CD16a. Also provided are methods of using the disclosed anti-CD16a antibodies and antigen-binding fragments thereof for reducing CD16a shedding from immune cells. Further provided are methods of using the disclosed anti-CD16a antibodies and antigen-binding fragments thereof for the treatment of cancer.

### CROSS-REFERENCE TO RELATED APPLICATIONS

This application claims the benefit of priority to U.S. Provisional Patent Application No. 63/369,720 filed on Jul. 28, 2022, the disclosure of which is hereby incorporated by reference herein in its entirety.

### REFERENCE TO AN ELECTRONIC SEQUENCE LISTING

The contents of the electronic sequence listing (SeqList_084284-00270.xml; Size: 18,907 bytes; and Date of Creation: Jul. 14, 2023) is herein incorporated by reference in its entirety.

### FIELD OF THE INVENTION

The present invention relates generally to the field of molecular biology and medicine. More particularly, the invention provides monoclonal antibodies and antigen-binding fragments that bind to CD16a and therapeutic compositions thereof, as well as methods of using such antibodies, including for treating cancer.

### BACKGROUND

Fc receptors are essential for protection against infections and cancers, by mediating a connection between adaptive immunity (antibodies) and innate immunity (macrophages, natural killer cells). Several cancer therapeutics are antibodies that engage Fc receptors, which in turn promote anti-tumor responses. For example, cetuximab is an anti-epidermal growth factor receptor (EGFR) antibody that is used in the clinic to treat colorectal, head, and neck cancers. Cetuximab not only blocks EGFR but also induces antibody dependent cellular cytotoxicity (ADCC) and phagocytosis by natural killer cells (“NK cells”), and macrophages, respectively.

Antibody-dependent cellular cytotoxicity (ADCC) is exerted by immune cells (including NK cells) expressing surface Fcγ receptors (FcγRs) against cells coated with antibody, including virus-infected, transformed cells, or cancer cells labeled with a therapeutic antibody targeting a cancer antigen. ADCC begins with recognition of an antigen expressed on the target cell surface by specific immunoglobulins. The Fc domain of these antibodies is then bound by FcγRs expressed on immune effector cells, which triggers the release of cytotoxic granules towards the target cell or upregulates death receptors expression on the cell surface.

ADCC in NK cells is exclusively mediated by Fc 7 receptor IIIa (FcγRIIIa, also referred to as CD16a). CD16a is also expressed by a subset of human blood monocytes. CD16a works coordinately with two other Fc activating receptors (CD32a and CD64) to trigger antibody-dependent phagocytosis by macrophages.

CD16a is downregulated by proteolytic cleavage, which is a post-translational modification performed by metalloprotease ADAM17. The cleavage causes the shedding of CD16a from cellular surfaces, thus preventing receptor engagement and signal transduction. Since cleavage-mediated loss of CD16a “disarms” NK cells, and perhaps macrophages too, CD16a shedding is a significant problem in immunotherapy with ADCC-inducing antibodies.

To address the problem of disarmed NK cells, ADAM17 inhibitors have been developed to stop the shedding of CD16a by NK cells and promote ADCC against diverse tumor cells tagged by antibodies against surface antigens in vitro. However, ADAM17 cleaves not only CD16a, but also multiple types of surface proteins (e.g., CD62L), cytokines (e.g., TNF-α), cytokine receptors (e.g., IL-6R), and growth factors (e.g., AREG). As such, ADAM17 inhibitors would cause pleiotropic effects if administered in vivo due to inhibition of cleavage of multiple surface proteins beyond just CD16a. Consistent with that limitation, ADAM17-deficient mice die at an early stage of development. Likewise, human ADAM17 deficiency, which is fortunately rare, causes multiple abnormalities with most of patients dying at infancy or childhood.

As an alternative solution to the problem of CD16a shedding, a variant of CD16a has been engineered that is resistant to cleavage, such as the mutant CD16a S197P. Primary NK cells expressing CD16a S197P were found to display higher degrees of ADCC against rituximab-tagged Raji cells. Rituximab is an anti-CD20 antibody. Similarly, induced pluripotent stem cell (iPSC)-derived NK cells expressing CD16a S197P also exhibited higher degrees of ADCC against rituximab-tagged Raji cells and cetuximab-tagged Cal-27 cells, compared to the ADCC by CD16a wild-type iPSC NK cells. However, such adoptive cell therapy approaches are lacking in that they fail to take advantage of endogenous NK cells and macrophages, which naturally express and cleave wild type CD16a.

Thus, methods of inhibiting CD16a cleavage in a highly specific and ADAM17-independent manner that bypass the need for genetic engineering are urgently needed.

### SUMMARY OF THE INVENTION

Provided herein are antibodies and antigen-binding fragments thereof that bind to CD16a and that reduce CD16a cleavage, thus reducing CD16a shedding from CD16a expressing immune cells. Also provided are methods of using the antibodies to promote immune cell activity and to treat cancer.

In one aspect, provided is an antibody or antigen-binding fragment thereof which binds to CD16a, the antibody or antigen-binding fragment thereof comprising a heavy chain variable region and a light chain variable region;
wherein each of the heavy chain and the light chain variable regions comprises a CDR1, CDR2, and CDR3; and wherein:
(a) the sequence of CDR1H comprises sequence GYTFTSYW (SEQ ID NO: 1);
(b) the sequence of CDR2H comprises sequence IYPGSGST (SEQ ID NO:2);
(c) the sequence of CDR3H comprises sequence TMRYGGYYGYYFDY (SEQ ID NO:3);
(d) the sequence of CDR1L comprises sequence SSISSNY (SEQ ID NO:4);
(e) the sequence of CDR2L comprises sequence RTS; and
(f) the sequence of CDR3L comprises sequence QQGSSIPLT (SEQ ID NO:5).

In embodiments, the sequence of the heavy chain variable region comprises a sequence that is at least 90% identical to SEQ ID NO:6; and the sequence of the light chain variable region comprises a sequence that is at least 90% identical to SEQ ID NO:8. In embodiments, the sequence of the heavy chain variable region comprises a sequence that is at least 95% identical to SEQ ID NO:6; and the sequence of the light chain variable region comprises a sequence that is at least 95% identical to SEQ ID NO:8. In one embodiment, the sequence of the heavy chain variable region comprises SEQ ID NO:6; and the sequence of the light chain variable region comprises SEQ ID NO:8.

In some embodiments, the anti-CD16 antibody or antigen-binding fragment thereof is a chimeric antibody, a CDR-grafted antibody, or a humanized antibody or antigen-binding fragment thereof.

In some embodiments, the anti-CD16 antibody or antigen-binding fragment thereof is a multispecific or a bispecific antibody or antigen-binding fragment thereof.

In some embodiments, the anti-CD16 antibody or antigen-binding fragment thereof is an scFv, Fv, Fab′, Fab, F(ab′)₂, or a diabody.

In one embodiment, the anti-CD16 antibody or antigen-binding fragment thereof has isotype IgG1.

In one embodiment, the anti-CD16 antibody or antigen-binding fragment thereof contains D265A N297A (Kabat EU index numbering) substitutions in the constant region of the heavy chain.

In one embodiment, the anti-CD16 antibody or antigen-binding fragment thereof is deglycosylated.

In one embodiment, the anti-CD16 antibody or antigen-binding fragment thereof is conjugated to one or more of a cytotoxin, a fluorescent label and an imaging agent.

In one embodiment, the anti-CD16 antibody or antigen-binding fragment thereof binds to the same epitope on human CD16a as an anti-CD16a antibody or antigen-binding fragment thereof disclosed herein.

Provided herein is an isolated nucleic acid or pair of nucleic acids encoding an anti-CD16 antibody or antigen-binding fragment thereof disclosed herein. Also provided is a vector or pair of vectors comprising an isolated nucleic acid or pair of nucleic acids disclosed herein.

Provided herein is an isolated cell comprising a vector disclosed herein. Provided herein is an isolated cell expressing the antibody or antigen-binding fragment thereof disclosed herein.

Provided herein is a pharmaceutical composition comprising an antibody or antigen-binding fragment thereof disclosed herein and a pharmaceutically acceptable excipient.

Provided herein is a method of reducing CD16a shedding, the method comprising contacting a cell expressing CD16a with an anti-CD16 antibody or antigen-binding fragment thereof disclosed herein. In one embodiment, the cell expressing CD16a is a NK cell. In one embodiment, the cell expressing CD16a is a macrophage. Provided herein is a method of reducing CD16a shedding in a subject in need thereof, the method comprising administering to the subject an anti-CD16a antibody or antigen-binding fragment thereof disclosed herein. Provided herein is a method of increasing NK cell effector function towards cancer cells, the method comprising contacting an NK cell expressing CD16a with an anti-CD16 antibody or antigen-binding fragment thereof disclosed herein. Provided herein is a method of increasing NK cell-mediated killing of cancer cells, the method comprising contacting an NK cell expressing CD16a with an anti-CD16 antibody or antigen-binding fragment thereof disclosed herein. Provided herein is a method of increasing NK cell-mediated antibody-dependent cellular cytotoxicity (ADCC) towards cancer cells, the method comprising contacting an NK cell expressing CD16a with an anti-CD16 antibody or antigen-binding fragment thereof disclosed herein. In embodiments, the method further comprises contacting the cancer cells with a second antibody or antigen-binding fragment thereof, wherein the second antibody or antigen-binding fragment thereof binds a cancer antigen presented by the cancer cell.

Provided herein is a method of increasing NK cell-driven immunity, the method comprising administering to a subject in need thereof an anti-CD16 antibody or antigen-binding fragment thereof disclosed herein. Provided herein is a method of increasing NK cell effector function towards cancer cells, the method comprising administering to a subject in need thereof an anti-CD16 antibody or antigen-binding fragment thereof disclosed herein. Provided herein is a method of increasing NK cell-mediated killing of cancer cells, the method comprising administering to a subject in need thereof an anti-CD16 antibody or antigen-binding fragment thereof disclosed herein. Provided herein is a method of increasing NK cell-mediated ADCC in a subject in need thereof, the method comprising administering to a subject in need thereof an anti-CD16 antibody or antigen-binding fragment thereof disclosed herein. Provided herein is a method of treating cancer in a subject in need thereof, the method comprising administering to the subject an anti-CD16 antibody or antigen-binding fragment thereof disclosed herein. In embodiments, the cancer is EGFR⁺ non-small cell lung cancer (NSCLC), Epidermoid carcinoma, EGFR⁺ colorectal carcinoma (CRC), Head and neck cancer, B-cell lymphoma, HER2⁺ breast cancer, Gastrointestinal cancers, PD-L1⁺ melanoma, Merkel cell and urothelial carcinomas, Squamous cell carcinoma of the lungs, Multiple myeloma, Neuroblastoma, acute or chronic myeloid leukemia, or myeloproliferative neoplasm. Provided herein is a method of reducing tumor growth in a subject in need thereof, the method comprising administering to the subject an anti-CD16 antibody or antigen-binding fragment thereof disclosed herein. Provided herein is a method of reducing tumor metastasis in a subject in need thereof, the method comprising administering to the subject an anti-CD16 antibody or antigen-binding fragment thereof disclosed herein. Provided herein is a method of reducing tumor-associated fibrosis in a subject in need thereof, the method comprising administering to the subject an anti-CD16 antibody or antigen-binding fragment thereof disclosed herein. Provided herein is a method of reducing cancer stemness in a subject in need thereof, the method comprising administering to an anti-CD16 antibody or antigen-binding fragment thereof disclosed herein. In embodiments, the method further comprises contacting the cancer cells with a second antibody or antigen-binding fragment thereof, wherein the second antibody or antigen-binding fragment thereof binds a cancer antigen presented by the cancer cell.

In embodiments, the second antibody or antigen-binding fragment thereof binds to a cancer antigen that is selected from the group consisting of CD19, CD30, CD33, CD123, CD47, CD133, BCMA, TEM8, EpCAM, ROR1, Folate Receptor, CD70, MAGE-1, MAGE-2, MAGE-3, MAGE A-10, MAGE-C2, MAGE-A12, CEA, tyrosinase, midkin, BAGE, CASP-8, β-catenin, CA-125, CDK-1, ESO-1, gp75, gp100, MART-1, MUC-1, MUM-1, p53, PAP, PSA, PSMA, ras, trp-1, TRP-1, TRP-2, IL13Ralpha, IL13Ralpha2, AIM-2, AIM-3, NY-ESO-1, C9orf112, SART1, SART2, SART3, BRAP, RTN4, GLEA2, TNKS2, KIAA0376, ING4, HSPH1, C13orf24, RBPSUH, C6orf153, NKTR, NSEP1, U2AF1L, CYNL2, TPR GOLGA, BMI1, COX-2, EGFRvIII, EZH2, LICAM, Livin, Livinβ, MRP-3, Nestin, OLIG2, ART1, ART4, B-cycline, Gli1, Cav-1, Cathepsin B, CD74, E-Cadherin, EphA2/Eck, Fra-1/Fosl 1, GAGE-1, Ganglioside, GnT-V, β1, 6-N, Ki67, Ku70/80, PROX1, PSCA, SOX10, SOXI1, Survivin, βhCG, WT1, mesothelin, melan-A, NY-BR-1, NY-CO-58, MN (gp250), telomerase, SSX-2, PRAME, PLK1, VEGF-A, VEGFR2, and Tie-2. In embodiments, the second antibody or antigen-binding fragment thereof binds to a cancer antigen that is selected from epidermal growth factor receptor (EGFR), CD20, HER-2, CD38, programmed death-ligand 1 (PD-L1) or GD2. In embodiments, the second antibody or antigen-binding fragment thereof is selected from the group consisting of cetuximab, rituximab, trastuzumab, avelumab, necitumumab, daratumumab, dinutuximab, zalutumumab, and nimotuzumab.

In some embodiments, the second antibody is administered concurrently with an anti-CD16a antibody or antigen-binding fragment thereof disclosed herein. In some embodiments, the second antibody is administered consecutively with an anti-CD16a antibody or antigen-binding fragment thereof disclosed herein.

Provided herein is a kit comprising an anti-CD16a antibody or antigen-binding fragment thereof disclosed herein and a second antibody or antigen-binding fragment thereof that binds a cancer antigen.

Provided herein is a method of producing an antibody or antigen-binding fragment thereof, the method comprising providing a cell expressing an anti-CD16 antibody or antigen-binding fragment thereof disclosed herein and isolating the antibody or antigen-binding fragment.

### BRIEF DESCRIPTION OF THE DRAWINGS

FIGS. 1A and 1B illustrate the selection of anti-CD16a antibody F9H4. FIG. 1A. ELISA with hybridoma supernatants or 1 μg/mL negative (isotype) or positive (3G8) control antibody, respectively. FIG. 1B. Flow cytometry analysis of surface CD16a on NK cells five hours after treatment with PMA plus 10 μg/mL isotype or 100 μL of hybridoma supernatants. Representative of three (FIG. 1A) and two (FIG. 1B) experiments are shown.

FIG. 2 shows the results of an ELISA demonstrating reactivity of F9H4 against the human CD16a protein.

FIGS. 3A, 3B, 3C, and 3D show that F9H4 inhibits CD16a shedding. Primary NK cells were treated with PMA to induce the shedding. FIG. 3A. Detection of soluble CD16a shed by NK cells. FIGS. 3B and 3C. Detection of surface CD16a by flow cytometry. FIG. 3D. Surface CD16a expression in human monocyte-derived macrophages, as analyzed by flow cytometry. Data represent three independent experiments (FIGS. 3A, 3B, 3C, and 3D), are mean+/−standard deviation (SD) of triplicates (FIG. 3C) or mean+/−standard error (SE) of triplicates (FIG. 3A) or quadruplicates (FIG. 3B, FIG. 3D), and were analyzed by non-linear regression (A, C, E) or one-way analysis of variance (ANOVA) with Dunnett's test (FIG. 3A). **p<0.01.

FIGS. 4A, 4B, 4C, 4D, 4E, and 4F illustrate the characterization of F9H4. FIG. 4A. An ELISA was performed with immobilized F9H4 or the positive controls, respectively. For detection, biotinylated antibodies against the indicated subclasses of murine IgG were used. From left to right: Anti-mIgG1, anti-mIgG2a, anti-mIgG2b, anti-mIgG3. FIG. 4B. Validation of a bead assay to assess CD16a binding to hIgG1. DANA is a mutant hIgG1 that does not bind CD16a and served as a control in the assay. Two left peaks (overlapping): uncoated and DANA mutant. Right peak: hIgG1. FIG. 4C. Bead assay for CD16a binding to hIgG1. hIgG1-coated beads were incubated with 1 μg/mL biotinylated CD16a plus the indicated antibodies, followed by incubation with PE-labelled streptavidin and analysis by flow cytometry. Peaks from left to right: 30, 10, 3, and 1 μg/mL. FIG. 4D. Jukart cells were engineered to express human CD16a and luciferase in response to CD16a engagement. Cells were treated overnight with the indicated antibodies, followed by addition of luciferin and analyses in plate reader. RLU=relative light units. FIG. 4E. Crystal structure of CD16a bound to Fc domain of hIgG1. Protein Data Bank 5YC5. FIG. 4F. ELISA whereby full length CD16a (indicated as “D1+D2”), D1, or D2 are immobilized in multi-well plates, followed by incubation with the indicated antibodies. Data represent two (FIG. 4D) or three (FIGS. 4A, 4B, 4C, and 4F) independent experiments, are mean+/−SE of quadruplicates (FIG. 4 D), and were analyzed by non-linear regression (FIGS. 4D and 4F).

FIGS. 5A, 5B, 5C, 5D, and 5E illustrate that anti-CD16a antibody F9H4 promotes NK cell-mediated ADCC in vitro. NK cells were co-cultured for four hours with A549 cells at 1:1 effector-to-target ratio (FIGS. 5A, 5B, and 5D.) or as indicated (FIG. 5C) in the presence of the indicated antibodies, followed by flow cytometry analyses. NK cells were identified as human CD45⁺ alive single cells. FIG. 5A. Analysis of CD107a externalization by NK cells. FIG. 5B. Analysis of intracellular interferon γ in NK cells. FIG. 5C. Analysis of A549 cells killed by NK cells, based on the labelling with 7-AAD and correction, by subtraction, for dead A549 cells in the absence of NK cells. FIG. 5D. CD107a externalization assay with additional control groups, as indicated. Left: Isotype: Right: F9H4. FIG. 5E. Expression of CD16a by NK cells after four hours co-culture with A549 (or NK cell alone) in the presence of the indicated antibodies. Left: Isotype: Right: F9H4. Data are mean+/−SD of triplicates (FIGS. 5A, 5B, 5D), or SE of triplicates (FIG. 5C), or SD of quadruplicates (FIG. 5E). Data represent three independent experiments (FIGS. 5A, 5B, 5C, 5D, and 5E). Data were analyzed by non-linear regression (FIGS. 5A and 5B) or two-way ANOVA with Bonferroni's test (FIGS. 5A, 5B, 5C, 5D, and 5E). *p<0.05, ***p<0.001.

FIG. 6 illustrates that a combination of F9H4 and cetuximab inhibits human lung cancer. Quantification of A549 tumors in the lungs of hIL-15 NOG mice reconstituted with human NK cells. Male 6-8-week-old hIL-15 mice were inoculated intravenously with 10⁶ human NK cells from HDs, and one week later they were inoculated intravenously with 1.5×10⁶ A549 cells. On days 1, 2, and once per week after A549 inoculation, the mice were treated with 0.1 mg of each one of the indicated antibodies. Analyses of tumors were done on day 21 by histopathology, as in FIG. 6B. Data are mean+/−SD of ten mice per group (FIG. 6C). Data are pooled of two independent experiments (FIG. 6C). Data were analyzed two-way ANOVA with Bonferroni's test. *p<0.05, ***p<0.001.

FIGS. 7A, 7B, and 7C illustrate the properties of chF9H4-DANA, a chimeric antibody in which the variable and the CH1 region of F9H4 is fused to the CH2 and CH3 regions of hIgG1 comprising a D265A N297A (DANA) mutation (labeled as “F9H4-hIG1-DANA” in FIGS. 7A, 7B, and 7C). FIG. 7A. The indicated antibodies were immobilized in ELISA plates and detected by biotinylated human CD64 protein, which was added in the indicated concentrations. Highest absorbance for hIgG wild type. FIG. 7B. ELISA for reactivity of antibodies against human CD16a. Highest absorbance for F9H4-hIG1-DANA. FIG. 7C. Inhibition of CD16a shedding assay with primary NK cells that were treated with 30 ng/mL PMA to induce CD16a shedding. Highest absorbance for F9H4-hIG1-DANA. Data represent three independent experiments (FIGS. 7A, 7B, and 7C) and mean+/−SD of triplicates even though the SD bars are so small that cannot be seen (FIG. 7C).

### DETAILED DESCRIPTION OF THE INVENTION

### Antibodies

The term “antibody” is used in the broadest sense and includes monoclonal antibodies (including full length or intact monoclonal antibodies), polyclonal antibodies, multivalent antibodies, multispecific antibodies (e.g., bispecific antibodies), antibody fragments, and antigen-binding portions thereof (e.g., paratopes, CDRs), so long as they exhibit the desired biological activity and specificity.

As used herein, “antibody variable domain” refers to the portions of the light and heavy chains of antibody molecules that include amino acid sequences of Complementarity Determining Regions (CDRs; i.e., CDR1, CDR2, and CDR3), and Framework Regions (FRs). V_{H} refers to the variable domain of the heavy chain. V_{L} refers to the variable domain of the light chain. The amino acid positions assigned to CDRs and FRs may be defined, for example, according to Kabat or according to Chothia. The term “framework regions” (FR) refers to those variable domain residues other than the CDR residues.

As used herein, the term “Complementarity Determining Regions” (CDRs) refers to portions of an antibody variable domain that are (typically) involved in antigen binding. Each variable domain typically has three CDR regions identified as CDR1, CDR2 and CDR3. Each CDR can comprise amino acid residues from a CDR as defined by e.g., Kabat (i.e., about residues 24-34 (L1), 50-56 (L2) and 89-97 (L3) in the light chain variable domain and 31-35 (H1), 50-65 (H2) and 95-102 (H3) in the heavy chain variable domain (Kabat et al., Sequences of Proteins of Immunological Interest, 5th Ed. Public Health Service, National Institutes of Health, Bethesda, Md. (1987, 1991)). Each CDR can also comprise amino acid residues from a “hypervariable loop” (i.e., about residues 26-32 (LI), 50-52 (L2) and 91-96 (L3) in the light chain variable domain and 26-32 (H1), 53-55 (H2) and 96-101 (H3) in the heavy chain variable domain (Chothia & Lesk 196 J. Mol. Biol. 901 (1987)). In some instances, a CDR can include amino acids from both a CDR region defined according to Kabat and a hypervariable loop. The Kabat residue designations do not always correspond directly with the linear numbering of the amino acid residues (primary amino acid sequence). The actual linear amino acid sequence may contain fewer or additional amino acids than in the strict Kabat numbering corresponding to a shortening of, or insertion into, a structural component, whether framework or CDR, of the basic variable domain structure. The correct Kabat numbering of residues may be determined for a given antibody or antigen-binding fragment thereof by alignment of residues of homology in the sequence of the antibody or antigen-binding fragment thereof with a “standard” Kabat numbered sequence. Alternatively, a CDR can be defined according to the ImMunoGeneTics (IMGT) system (Lefranc, M.-P. et al., Dev. Comp. Immunol., 27, 55-77 (2003)).

As used herein, the term “constant region” refers to a region of an immunoglobulin light chain or heavy chain that is distinct from the variable region. The constant domain of the heavy chain generally comprises at least one of: a CH1 domain, a hinge (e.g., upper, middle, and/or lower hinge region), a CH2 domain, and a CH3 domain. For example, an antibody described herein may comprise a polypeptide comprising a CH1 domain; a polypeptide comprising a CH1 domain, at least a portion of a hinge domain, and a CH2 domain; a polypeptide comprising a CH1 domain and a CH3 domain; a polypeptide comprising a CH1 domain, at least a portion of a hinge domain, and a CH3 domain, or a polypeptide comprising a CH1 domain, at least a portion of a hinge domain, a CH2 domain, and a CH3 domain. In some embodiments, a polypeptide comprises a polypeptide chain comprising a CH3 domain. The constant domain of a light chain can be a kappa (κ) or lambda (λ) constant region. However, it will be understood by one of ordinary skill in the art that these constant domains (e.g., the heavy chain or light chain) may be modified such that they vary in amino acid sequence from the naturally occurring immunoglobulin molecule. HC refers to the heavy chain, including the V_{H} and the constant region. LC refers to the light chain, including the V_{L} and the constant region.

As used herein, the term “Fc region” or “Fc portion” refers to the C terminal region of an immunoglobulin heavy chain. The Fc region can be a native-sequence Fc region or a non-naturally occurring variant Fc region. Generally, the Fc region of an immunoglobulin comprises constant domains CH2 and CH3. Although the boundaries of the Fe region can vary, in some embodiments, the human IgG heavy chain Fc region can be defined to extend from an amino acid residue at position C226 or from P230 to the carboxy terminus thereof. In some embodiments, the “CH2 domain” of a human IgG Fc region, usually extends from about amino acid residue 231 to about amino acid residue 340. In some embodiments, N-linked carbohydrate chains are interposed between the two CH2 domains of an intact native IgG molecule. In some embodiments, the CH3 domain” of a human IgG Fc region comprises residues C-terminal to the CH2 domain, e.g., from about amino acid residue 341 to about amino acid residue 447 of the Fc region.

In one embodiment, the anti-CD16a antibody or antigen-binding fragment thereof provided herein comprises a heavy variable chain comprising three CDRs, wherein:
a) the sequence of CDR1H comprises sequence GYTFTSYW (SEQ ID NO: 1);
b) the sequence of CDR2H comprises sequence IYPGSGST (SEQ ID NO:2); and
c) the sequence of CDR3H comprises sequence TMRYGGYYGYYFDY (SEQ ID NO:3);

In one embodiment, the Anti-CD16a antibody or antigen-binding fragment thereof provided herein comprises a light variable chain comprising three CDRs, wherein:
a) the sequence of CDR1L comprises sequence SSISSNY (SEQ ID NO:4);
b) the sequence of CDR2L comprises sequence RTS; and
c) the sequence of CDR3L comprises sequence QQGSSIPLT (SEQ ID NO:5).

In one embodiment, the Anti-CD16a antibody or antigen-binding fragment thereof provided herein comprises six CDRs, wherein:
a) the sequence of CDR1H comprises sequence GYTFTSYW (SEQ ID NO:1);
b) the sequence of CDR2H comprises sequence IYPGSGST (SEQ ID NO:2);
c) the sequence of CDR3H comprises sequence TMRYGGYYGYYFDY (SEQ ID NO:3);
d) the sequence of CDR1L comprises sequence SSISSNY (SEQ ID NO:4);
e) the sequence of CDR2L comprises sequence RTS; and
f) the sequence of CDR3L comprises sequence QQGSSIPLT (SEQ ID NO:5).
   According to certain embodiments, the contemplated antibodies and antigen-binding fragments thereof also feature humanized frameworks for reduced immunogenicity. In certain embodiments, the CDRs of the contemplated antibody or antigen-binding fragment thereof are located in frameworks obtained from a human antibody or antigen-binding fragment thereof. In other embodiments, surface-exposed framework residues of the contemplated antibody or antigen-binding fragment thereof are replaced with framework residues of a human antibody or antigen-binding fragment thereof. The CDRs may also be located in murine or humanized frameworks linked to human constant regions (i.e., chimeric antibodies). In one embodiment, the antibody or antigen-binding fragment thereof comprises a murine variable region and human constant regions. In one embodiment, the antibody or antigen-binding fragment thereof comprises a murine variable region, a murine CH1 region and human CH2 and CH3 constant regions. In one embodiment, the CDRs of a contemplated antibody or antigen-binding fragment thereof are located in frameworks that are a composite of two or more human antibodies. In such embodiments, the contemplated antibodies or antigen-binding fragments thereof comprise two or more sequence segments (“composites”) derived from V-regions of unrelated human antibodies that are selected to maintain monoclonal antibody sequences important for antigen binding of the starting precursor anti-human CD16a monoclonal antibody, and which have all been filtered for the presence of potential T cell epitopes using “in silico tools” (see, e.g., Holgate & Baker, IDrugs. 2009 April; 12(4):233-7). The close fit of human sequence segments with all sections of the starting antibody V regions and the elimination of CD4⁺ T cell epitopes prior to synthesis of the antibody or antigen-binding fragment thereof allow this technology to circumvent immunogenicity while maintaining optimal affinity and specificity through the prior analysis of sequences necessary for antigen-specificity (Holgate & Baker, 2009).
   Also provided herein are variable heavy chain and variable light chain sequences (as well as pairing thereof) that comprise sequences that are similar, but not identical to the variable heavy chain and variable light chains disclosed in SEQ ID NOs:6 or 8.

In one embodiment, the anti-CD16a antibody or antigen-binding fragment thereof comprises a heavy chain variable domain comprising a sequence that is at least 80%, at least 85%, at least 90%, at least 95%, at least 96%, at least 97%, at least 98%, or at least 99% identical to SEQ ID NO:6. In one embodiment, the anti-CD16a antibody or antigen-binding fragment thereof comprises a light chain variable domain comprising a sequence that is at least 80%, at least 85%, at least 90%, at least 95%, at least 96%, at least 97%, at least 98%, or at least 99% identical to SEQ ID NO:8.

In one embodiment, the anti-CD16a antibody or antigen-binding fragment thereof comprises
(i) a heavy chain variable domain comprising a sequence that is at least 80%, at least 85%, at least 90%, at least 95%, at least 96%, at least 97%, at least 98%, or at least 99% identical to SEQ ID NO:6; and
(ii) a light chain variable domain comprising a sequence that is at least 80%, at least 85%, at least 90%, at least 95%, at least 96%, at least 97%, at least 98%, or at least 99% identical to SEQ ID NO:8.

In one embodiment, the anti-CD16a antibody or antigen-binding fragment thereof comprises
(i) a heavy chain variable domain comprising SEQ ID NO:6; and/or
(ii) a light chain variable domain comprising SEQ ID NO:8.

In one embodiment, the anti-CD16a antibody or antigen-binding fragment thereof comprises
(i) a heavy chain variable domain comprising SEQ ID NO:6; and
(ii) a light chain variable domain comprising SEQ ID NO:8.

In one embodiment, the anti-CD16a antibody or antigen-binding fragment thereof comprises a heavy chain comprising a sequence that is at least 80%, at least 85%, at least 90%, at least 95%, at least 96%, at least 97%, at least 98%, or at least 99% identical to SEQ ID NO:7 or SEQ ID NO:15. In one embodiment, the anti-CD16a antibody or antigen-binding fragment thereof comprises a light chain comprising a sequence that is at least 80%, at least 85%, at least 90%, at least 95%, at least 96%, at least 97%, at least 98%, or at least 99% identical to SEQ ID NO:9.

In one embodiment, the anti-CD16a antibody or antigen-binding fragment thereof comprises
(i) a heavy chain comprising a sequence that is at least 80%, at least 85%, at least 90%, at least 95%, at least 96%, at least 97%, at least 98%, or at least 99% identical to SEQ ID NO:7; and
(ii) a light chain comprising a sequence that is at least 80%, at least 85%, at least 90%, at least 95%, at least 96%, at least 97%, at least 98%, or at least 99% identical to SEQ ID NO:9.

In one embodiment, the anti-CD16a antibody or antigen-binding fragment thereof comprises
(i) a heavy chain comprising SEQ ID NO:7; and/or
(ii) a light chain comprising SEQ ID NO:9.

In one embodiment, the anti-CD16a antibody or antigen-binding fragment thereof comprises
(i) a heavy chain comprising SEQ ID NO:7; and
(ii) a light chain comprising SEQ ID NO:9.

In one embodiment, the anti-CD16a antibody or antigen-binding fragment thereof comprises
(i) a heavy chain comprising a sequence that is at least 80%, at least 85%, at least 90%, at least 95%, at least 96%, at least 97%, at least 98%, or at least 99% identical to SEQ ID NO:15; and
(ii) a light chain comprising a sequence that is at least 80%, at least 85%, at least 90%, at least 95%, at least 96%, at least 97%, at least 98%, or at least 99% identical to SEQ ID NO:9.

In one embodiment, the anti-CD16a antibody or antigen-binding fragment thereof comprises
(i) a heavy chain comprising SEQ ID NO:15; and/or
(ii) a light chain comprising SEQ ID NO:9.

In one embodiment, the anti-CD16a antibody or antigen-binding fragment thereof comprises
(i) a heavy chain comprising SEQ ID NO:15; and
(ii) a light chain comprising SEQ ID NO:9.

As used herein, the term “identity” refers to sequence identity between two nucleic acid molecules or polypeptides. Identity can be determined by comparing a position in each sequence which may be aligned for purposes of comparison. For example, when a position in the compared nucleotide sequence is occupied by the same base, then the molecules are identical at that position. A degree identity between nucleic acid or amino acid sequences is a function of the number of identical or matching nucleotides or amino acids at shared positions. For example, polypeptides having at least 85%, 90%, 95%, 98%, or 99% identity to specific polypeptides described herein and preferably exhibiting substantially the same functions, as well as polynucleotides encoding such polypeptides, are contemplated. Methods and computer programs for determining both sequence identity and similarity are publicly available, including, but not limited to, the GCG program package (Devereux et al., Nucleic Acids Research 12: 387, 1984), BLASTP, BLASTN, FASTA (Altschul et al., J. Mol. Biol. 215:403 (1990), and the ALIGN program (version 2.0). The well-known Smith Waterman algorithm may also be used to determine similarity. The BLAST program is publicly available from NCBI and other sources (BLAST Manual, Altschul, et al., NCBI NLM NIH, Bethesda, Md. 20894; BLAST 2.0 at http://www.ncbi.nlm.nih.gov/blast/). In comparing sequences, these methods account for various substitutions, deletions, and other modifications.

In one embodiment, provided is an CD16a antibody or antigen-binding fragment thereof comprises
(i) a heavy chain variable domain comprising a sequence that is at least 80%, at least 85%, at least 90%, at least 95%, at least 96%, at least 97%, at least 98%, or at least 99% identical to SEQ ID NO:6;
(ii) a light chain variable domain comprising a sequence that is least 80%, at least 85%, at least 90%, at least 95%, at least 96%, at least 97%, at least 98%, or at least 99% identical to SEQ ID NO:8; and
(iii) six CDRs, wherein:
   a) the sequence of CDR1H comprises sequence GYTFTSYW (SEQ ID NO: 1);
   b) the sequence of CDR2H comprises sequence IYPGSGST (SEQ ID NO:2);
   c) the sequence of CDR3H comprises sequence TMRYGGYYGYYFDY (SEQ ID NO:3);
   d) the sequence of CDR1L comprises sequence SSISSNY (SEQ ID NO:4);
   e) the sequence of CDR2L comprises sequence RTS; and
   f) the sequence of CDR3L comprises sequence QQGSSIPLT (SEQ ID NO:5).

In one embodiment, provided is an CD16a antibody or antigen-binding fragment thereof comprises
(i) a heavy chain comprising a sequence that is at least 80%, at least 85%, at least 90%, at least 95%, at least 96%, at least 97%, at least 98%, or at least 99% identical to SEQ ID NO:7;
(ii) a light chain comprising a sequence that is least 80%, at least 85%, at least 90%, at least 95%, at least 96%, at least 97%, at least 98%, or at least 99% identical to SEQ ID NO:9; and
(iii) six CDRs, wherein:
   a) the sequence of CDR1H comprises sequence GYTFTSYW (SEQ ID NO:1);
   b) the sequence of CDR2H comprises sequence IYPGSGST (SEQ ID NO:2);
   c) the sequence of CDR3H comprises sequence TMRYGGYYGYYFDY (SEQ ID NO:3);
   d) the sequence of CDR1L comprises sequence SSISSNY (SEQ ID NO:4);
   e) the sequence of CDR2L comprises sequence RTS; and
   f) the sequence of CDR3L comprises sequence QQGSSIPLT (SEQ ID NO:5).

In some embodiments of the aspects described herein, amino acid sequence modification(s) of the antibodies or antigen-binding fragments thereof that bind to CD16a described herein are contemplated. Amino acid sequence variants of the antibody or antigen-binding fragment thereof are prepared by introducing appropriate nucleotide changes into the nucleic acid encoding the antibody or antigen-binding fragment thereof, or by peptide synthesis. Such modifications include, for example, deletions from, and/or insertions into and/or substitutions of, residues within the amino acid sequences of the antibody or antigen-binding fragment thereof. Any combination of deletion, insertion, and substitution is made to arrive at the final construct, provided that the final construct possesses the desired characteristics, e.g., binding specificity, inhibition of biological activity.

One type of variant is a conservative amino acid substitution variant. These variants have at least one amino acid residue in the antibody or antigen-binding fragment thereof replaced by a different residue that has similar side chain properties. Amino acids can be grouped according to similarities in the properties of their side chains (see Lehninger, BIOCHEMISTRY (2nd ed., Worth Publishers, New York, 1975): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H).

As such, a non-limiting example for a conservative amino acid substitution is one that replaces a non-polar amino acid with another non-polar amino acid.

Alternatively, naturally occurring residues can be divided into groups based on common side-chain properties:
(1) hydrophobic: Ala (A), Val (V), Leu (L), Ile (I), Met (M);
(2) neutral hydrophilic: Ser (S), Thr (T), Cys (C), Asn (N), Gln (Q);
(3) acidic: Asp (D), Glu (E);
(4) basic: Lys (K), Arg (R), His (H);
(5) residues that influence chain orientation: Gly (G), Pro (P);
(6) aromatic: Phe (F), Trp (W), Tyr (Y).
   As such, a non-limiting example for a conservative amino acid substitution is one that replaces a hydrophobic amino acid with another hydrophobic amino acid.

Further contemplated are amino acid sequence insertions, which can include amino- and/or carboxyl-terminal fusions ranging in length from one residue to polypeptides containing a hundred or more residues, as well as intrasequence insertions of single or multiple amino acid residues. Examples of terminal insertions include an antibody or antigen-binding fragment thereof with an N-terminal methionyl residue or the antibody or antigen-binding fragment thereof fused to a cytotoxic polypeptide. Other insertional variants of the antibody or antigen-binding fragment thereof include the fusion to the N- or C-terminus of the antibody or antigen-binding fragment thereof to an enzyme or a polypeptide which increases the serum half-life of the antibody or antigen-binding fragment thereof, such as, for example, biotin.

Any cysteine residue not involved in maintaining the proper conformation of the antibodies or antigen-binding fragments thereof that bind to CD16a also can be substituted, for example with a serine or an alanine, to improve the oxidative stability of the molecule and prevent aberrant crosslinking.

Conversely, cysteine bond(s) can be added to the antibody or antigen-binding fragment thereof to improve its stability (particularly where the antibody or antigen-binding fragment thereof is an antibody fragment such as an Fv fragment).

In some embodiments, the antibodies or antigen-binding fragments thereof described herein have amino acid alterations that alter the original glycosylation pattern of the antibody or antigen-binding fragment thereof. By “altering the original glycosylation pattern” is meant deleting one or more carbohydrate moieties found in the antibody or antigen-binding fragment thereof, and/or adding one or more glycosylation sites that are not present in the antibody or antigen-binding fragment thereof. Glycosylation of antibodies is typically either N-linked or 0-linked. N-linked refers to the attachment of the carbohydrate moiety to the side chain of an asparagine residue. The tripeptide sequences asparagine-X-serine and asparagine-X-threonine, wherein X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine can also be used. Addition of glycosylation sites to the antibodies or antigen-binding fragments thereof that bind to CD16a is accomplished by altering the amino acid sequence such that it contains one or more of the above-described tripeptide sequences (for N-linked glycosylation sites). The alteration can also be made by the addition of, or substitution by, one or more serine or threonine residues to the sequence of the original antibody or antigen-binding fragment thereof (for O-linked glycosylation sites).

In some embodiments, the CD16a antibodies or antigen-binding fragments thereof provided herein are deglycosylated or aglycosylated. Where the antibody or antigen-binding fragment thereof comprises an Fc region, the carbohydrate(s) attached thereto can be altered. For example, antibodies with a mature carbohydrate structure that lacks fucose attached to an Fc region of the antibody or antigen-binding fragment thereof are described. See, e.g., U.S. Patent Pubs. No. 2003/0157108; No. 2004/0093621. Antibodies with a bisecting N-acetylglucosamine (GlcNAc) in the carbohydrate attached to an Fc region of the antibody or antigen-binding fragment thereof are referenced in WO 03/011878; U.S. Pat. No. 6,602,684. Antibodies with at least one galactose residue in the oligosaccharide attached to an Fc region of the antibody or antigen-binding fragment thereof are reported in WO 97/30087. See also WO 98/58964; WO 99/22764 concerning antibodies with altered carbohydrate attached to the Fc region thereof.

In some embodiments, the antibodies or antigen-binding fragments thereof disclosed herein are modified to exhibit effector function reduction or elimination. This can, for example, be accomplished by: (i) reduction or elimination of wild-type mammalian glycosylation of the antibody, (for example, by producing the antibody in an environment where such glycosylation cannot occur, by mutating one or more carbohydrate attachment points such that the antibody cannot be glycosylated, or by chemically or enzymatically removing one or more carbohydrates from the antibody after it has been glycosylated); (ii) by reduction or elimination of the Fc receptor-binding capability of the antibody (for example, by mutation of the Fc region, by deletion within the Fc region or elimination of the Fc region); or (iii) by utilization of an antibody isotype known to have minimal or no effector function (i.e., including but not limited to IgG4). In some embodiments, the heavy chain constant region has one or more of the following mutations: S228P; N297Q; and T299A (Kabat EU index numbering). In some embodiments, the heavy chain constant region has one or more of the following mutations: L234A, L235A, and P329G (Kabat EU index numbering). In some embodiments, the heavy chain constant region has a D265A N297A mutation (Kabat EU index numbering). The location of the DANA mutation is shown in Table 3 (see SEQ ID NO:13 as an example). In some embodiments, the heavy chain constant region does not have a D265A N297A mutation.

Antibodies with improved binding to the neonatal Fc receptor (FcRn), and increased half-lives, are described in WO 00/42072 and U.S. Patent Pub. No. 2005/0014934. These antibodies comprise an Fc region with one or more substitutions therein which improve binding of the Fc region to FcRn. For example, the Fc region can have substitutions at one or more of positions 238, 250, 256, 265, 272, 286, 303, 305, 307, 311, 312, 314, 317, 340, 356, 360, 362, 376, 378, 380, 382, 413, 424, 428 or 434 (Eu numbering of residues). The preferred Fc region-comprising an antibody variant with improved FcRn binding comprises amino acid substitutions at one, two or three of positions 307, 380 and 434 of the Fc region thereof (Eu numbering of residues). In one embodiment, the antibody or antigen-binding fragment thereof has a 307 and a 434 mutation.

Engineered antibodies that bind to CD16a with three or more (e.g., four) functional antigen binding sites are also contemplated. See, e.g., U.S. Patent Pub. No. US 2002/0004587.

### Antibody Fragments and Types

In some embodiments of the aspects described herein, the anti-CD16a antibody fragment is a Fab fragment, which comprises or consist essentially a variable (V_{L}) and constant (C_{L}) domain of the light chain and a variable domain (V_{H}) and the first constant domain (C_{H}1) of the heavy chain.

In some embodiments of the aspects described herein, the anti-CD16a antibody fragment is a Fab′ fragment, which refers to a Fab fragment having one or more cysteine residues at the C-terminus of the C_{H}1 domain.

In some embodiments of the aspects described herein, the anti-CD16a antibody fragment is an Fd fragment comprising or consisting essentially of V_{H} and C_{H}1 domains.

In some embodiments of the aspects described herein, the anti-CD16a antibody portion is an Fd′ fragment comprising V_{H} and C_{H}1 domains and one or more cysteine residues at the C-terminus of the C_{H}1 domain.

Single-chain Fv or scFv antibody fragments comprise or consist essentially of the V_{H} and V_{L} domains of antibody, such that these domains are present in a single polypeptide chain. Generally, an Fv polypeptide further comprises a polypeptide linker between the V_{H} and V_{L} domains, which allows the scFv to form the desired structure for antigen binding. See, for example, Pluckthun, 113 Pharmacology Monoclonal Antibodies 269 (Rosenburg & Moore, eds., Springer-Verlag, New York, 1994). Accordingly, in some embodiments of the aspects described herein, the anti-CD16a antibody fragment is a Fv fragment comprising or consisting essentially of the V_{L} and V_{H} domains of a single arm of an antibody.

In some embodiments of the aspects described herein, the anti-CD16a antibody portion is a diabody comprising two antigen binding sites, comprising a heavy chain variable domain (V_{H}) connected to a light chain variable domain (V_{L}) in the same polypeptide chain.

In some embodiments of the aspects described herein, the anti-CD16a antibody portion is a dAb fragment comprising or consisting essentially of a V_{H} domain.

In some embodiments of the aspects described herein, the anti-CD16a antibody portion is a F(ab′)2 fragment, which comprises a bivalent fragment comprising two Fab′ fragments linked by a disulfide bridge at the hinge region.

Linear antibodies refer to the antibodies as described in Zapata et al., Protein Engin., 8(10):1057-1062 (1995). Briefly, these antibodies comprise a pair of tandem Fd segments (V_{H}-C_{H}1-V_{H}-C_{H}1), which, together with complementary light chain polypeptides, form a pair of antigen binding regions. Linear antibodies can be bispecific or monospecific. In some embodiments of the aspects described herein, the anti-CD16a antibody fragment is a linear antibody comprising a pair of tandem Fd segments (V_{H}-C_{H}1-V_{H}-C_{H}1) which, together with complementary light chain polypeptides, form a pair of antigen binding regions.

Various techniques have been developed and are available for the production of antibody fragments. Traditionally, these fragments were derived via proteolytic digestion of intact antibodies. See, e.g., Morimoto et al., 24 J. Biochem. Biophys. Meths. 107 (1992); Brennan et al., 229 Science 81 (1985). However, these fragments can now be produced directly by recombinant host cells. For example, antibody fragments can be isolated from the antibody phage libraries discussed herein. Alternatively, Fab′-SH fragments can be directly recovered from E. coli and chemically coupled to form F(ab′)2 fragments (Carter et al., 1992). According to another approach, F(ab′)2 fragments can be isolated directly from recombinant host cell culture. Other techniques for the production of antibody fragments will be apparent to the skilled practitioner. In other embodiments, the antibody fragment of choice is a single chain Fv fragment (scFv).

Contemplated antibodies or antigen-binding fragments may have all types of constant regions, including IgM, IgG, IgD, and IgE, and any isotype, including IgG1, IgG2, IgG3, and IgG4. In one embodiment, the human isotype IgG1 is used. In another embodiment, the human isotype IgG4 is used. Light chain constant regions can be λ or κ. The antibody or antigen-binding fragment thereof may comprise sequences from more than one class or isotype.

### Antibody Binding

Provided herein are antibodies or antigen-binding fragments that bind to CD16. The extracellular region of CD16a has two immunoglobulin (Ig)-like domains, which are called D1 (the membrane distal domain) and D2 (the membrane proximal domain). D1 and D2 are connected by a hinge, and they form hydrogen bonds and Van der Waals interactions by several hydrophobic amino acids that cause D1 to bend over D2 at an angle of 52 degrees. D2 binds the Fc domain of hIgG1, whereas D1 does not participate in that interaction. Provided herein is an anti-CD16 antibody or antigen-binding fragment that binds the D1 domain.

As used herein, “binding” of an antibody or antigen binding fragment thereof to CD16a, an epitope on CD16a, or, in certain embodiments described below, particular residues on CD16a, includes the selective interaction of the antibody or antigen binding fragment thereof with CD16a. Binding therefore includes, e.g., primary and secondary interactions including hydrogen bonds, ionic interactions, salt bridges, as well as hydrophilic and hydrophobic interactions.

In certain embodiments, the CD16a antibodies or antigen-binding fragments thereof described herein bind to CD16a with a K_{D} of 10⁻⁵ to 10⁻¹² mol/l, 10⁻⁶ to 10⁻¹² mol/l, 10⁻⁶ to 10⁻⁹ mol/l, 10⁻⁷ to 10⁻¹² mol/l, 10⁻¹ to 10⁻² mol/l, 10⁻⁹ to 10⁻¹² mol/l, 10⁻¹⁰ to 10⁻¹² mol/l, or 10⁻¹¹ to 10⁻¹² mol/l. In other embodiments, the CD16a antibodies or antigen-binding fragments thereof described herein bind to CD16a with a K_{D} of 10⁻⁵ to 10⁻¹¹ mol/l, 10⁶ to 10⁻¹¹ mol/l, 10⁻⁷ to 10¹¹ mol/l, 10⁻⁸ to 10⁻¹¹ mol/l, 10⁻⁹ to 10⁻¹¹ mol/l, or 10⁻¹⁰ to 10⁻¹¹ mol/l. In other embodiments, the CD16a antibodies or antigen-binding fragments thereof described herein bind to CD16a with a K_{D} of 10⁻⁵ to 10⁻¹⁰ mol/l, 10⁻⁶ to 10¹⁰ mol/l, 10⁻⁷ to 10⁻¹⁰ mol/l, 10⁻⁶ to 10⁻¹⁰ mol/l, or 10⁻⁷ to 10⁻¹⁰ mol/l. In other embodiments, the CD16a antibodies or antigen-binding fragments thereof described herein bind to CD16a with a K_{D} of 10⁻⁵ to 10⁻⁸ mol/l, 10⁻⁶ to 10⁻⁸ mol/l, or 10⁻⁷ to 10⁻⁸ mol/l.

The term “specificity” herein refers to the ability of an antibody or antigen-binding fragment thereof, such as an anti-CD16a antibody or antigen-binding fragment thereof, to recognize an epitope within CD16a, while only having little or no detectable reactivity with other portions of CD16a. Antibodies with low specificity bind to multiple epitopes. Specificity can be relatively determined by competition assays or by epitope identification/characterization techniques described herein or their equivalents known in the art.

As used herein, an “epitope” can be formed both from contiguous amino acids, or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5, about 9, or about 8-10 amino acids in a particular spatial conformation. An “epitope” includes the unit of structure conventionally bound by an immunoglobulin V_{H}/V_{L} pair. Epitopes define the minimum binding site for an antibody or antigen-binding fragment thereof, and thus represent the target of specificity of an antibody or antigen-binding fragment thereof. In the case of a single domain antibody, an epitope represents the unit of structure bound by a variable domain in isolation.

In a particular embodiment, the contemplated antibody or antigen-binding fragment specifically binds to the same epitope as antibody F9H4.

As used herein, a “blocking” antibody or an antibody “antagonist” is one that inhibits or reduces biological activity of the antigen to which it binds. For instance, competing, cross-blocking, and cross-blocked antibodies can be identified using any suitable method known in the art, including competition ELISAs or BIACORE® assays where binding of the competing or cross-blocking antibody to CD16a prevents the binding of another anti-CD16a antibody. In one aspect, provided is an anti-CD16a antibody or antigen-binding fragment thereof that does not reduce CD16a activity/block CD16a.

In certain embodiments, not all CDRs are directly involved in binding to the antigen. In one embodiment, four out of six CDRs of the anti-CD16a antibody or antigen-binding fragment thereof make contact with the antigen. In one embodiment, five out of six CDRs of the anti-CD16a antibody or antigen-binding fragment thereof make contact with the antigen. In one embodiment, six out of six CDRs of the anti-CD16a antibody or antigen-binding fragment thereof make contact with the antigen.

The terms “selective” and “selectivity” herein refer to the preferential binding of an antibody or antigen-binding fragment thereof (i.e., an anti-CD16a antibody or antigen-binding fragment thereof), for a particular region, target, or peptide; typically, a region or epitope in CD16a, as opposed to one or more other biological molecules.

As used herein, “affinity”, represented by the equilibrium constant for the dissociation (K_{D}) of an antigen with an antigen-binding protein, is a measure of the binding strength between an antigenic determinant and an antigen-binding site on the antigen-binding protein, such as an antibody or antibody fragment thereof. The smaller the value of the K_{D}, the stronger the binding strength between an antigenic determinant and the antigen-binding molecule. Alternatively, the affinity can also be expressed as the affinity constant (K_{A}), which is 1/K_{D}). As will be clear to the skilled person, affinity can be determined in a manner known per se, depending on the specific antigen of interest.

### Antibody Conjugates

In some embodiments of the aspects described herein, the antibody or antigen-binding fragment thereof that bind to CD16a are conjugated to a functional moiety. Examples of useful functional moieties include, but are not limited to, a blocking moiety, a detectable moiety, a diagnostic moiety, a targeting moiety, and a therapeutic moiety.

Exemplary blocking moieties include moieties of sufficient steric bulk and/or charge such that reduced glycosylation occurs, for example, by blocking the ability of a glycosidase to glycosylate the antibody or antigen-binding fragment thereof. The blocking moiety may additionally or alternatively, reduce effector function, for example, by inhibiting the ability of the Fc region to bind a receptor or complement protein. Preferred blocking moieties include cysteine adducts and PEG moieties.

In a preferred embodiment, the blocking moiety is a cysteine, preferably a cysteine that has associated with a free cysteine, e.g., during or subsequent to the translation of the Fc containing polypeptide, e.g., in cell culture. Other blocking cysteine adducts include cystine, mixed disulfide adducts, or disulfide linkages.

In another preferred embodiment, the blocking moiety is a polyalkylene glycol moiety, for example, a PEG moiety and preferably a PEG-maleimide moiety. Preferred pegylation moieties (or related polymers) can be, for example, polyethylene glycol (“PEG”), polypropylene glycol (“PPG”), polyoxyethylated glycerol (“POG”) and other polyoxyethylated polyols, polyvinyl alcohol (“PVA”) and other polyalkylene oxides, polyoxyethylated sorbitol, or polyoxyethylated glucose. The polymer can be a homopolymer, a random or block copolymer, a terpolymer based on the monomers listed above, straight chain or branched, substituted or unsubstituted as long as it has at least one active sulfone moiety. The polymeric portion can be of any length or molecular weight, but these characteristics can affect the biological properties. Polymer average molecular weights particularly useful for decreasing clearance rates in pharmaceutical applications are in the range of 2,000 to 35,000 Daltons. In addition, if two groups are linked to the polymer, one at each end, the length of the polymer can impact upon the effective distance, and other spatial relationships, between the two groups. Thus, one skilled in the art can vary the length of the polymer to optimize or confer the desired biological activity. PEG is useful in biological applications for several reasons. PEG typically is clear, colorless, odorless, soluble in water, stable to heat, inert to many chemical agents, does not hydrolyze, and is nontoxic. Pegylation can improve pharmacokinetic performance of a molecule by increasing the molecule's apparent molecular weight. The increased apparent molecular weight reduces the rate of clearance from the body following subcutaneous or systemic administration. In many cases, pegylation can decrease antigenicity and immunogenicity. In addition, pegylation can increase the solubility of a biologically-active molecule.

Examples of detectable moieties which are useful in the methods and antibodies and antigen-binding fragments thereof contemplated by the invention include fluorescent moieties or labels, imaging agents, radioisotopic moieties, radiopaque moieties, and the like, e.g., detectable labels such as biotin, fluorophores, chromophores, spin resonance probes, or radiolabels. Exemplary fluorophores include fluorescent dyes (e.g., fluorescein, rhodamine, and the like) and other luminescent molecules (e.g., luminal). A fluorophore may be environmentally-sensitive such that its fluorescence changes if it is located close to one or more residues in the modified protein that undergo structural changes upon binding a substrate (e.g., dansyl probes). Exemplary radiolabels include small molecules containing atoms with one or more low sensitivity nuclei (¹³C, ¹⁵N, ²H, ¹²⁵, ¹²³I, ⁹⁹Tc, ⁴³K, ⁵²Fe, ⁶⁷Ga, ⁶⁸Ga, ¹¹¹In and the like). Other useful moieties are known in the art.

Examples of diagnostic moieties which are useful in the methods and antibodies and antigen-binding fragments thereof contemplated by the invention include detectable moieties suitable for revealing the presence of a disease or disorder. Typically, a diagnostic moiety allows for determining the presence, absence, or level of a molecule, for example, a target peptide, protein, or proteins, that is associated with a disease or disorder. Such diagnostics are also suitable for prognosing and/or diagnosing a disease or disorder and its progression.

Examples of therapeutic moieties which are useful in the methods and antibodies and antigen-binding fragments thereof contemplated by the invention include, for example, anti-inflammatory agents, anti-cancer agents, anti-neurodegenerative agents, or anti-infective agents. The functional moiety may also have one or more of the above-mentioned functions.

Exemplary therapeutic moieties include radionuclides with high-energy ionizing radiation that are capable of causing multiple strand breaks in nuclear DNA, and therefore suitable for inducing cell death (e.g., of a cancer). Exemplary high-energy radionuclides include: ⁹⁰Y, ¹²⁵I, ¹³¹I, ¹²³I, ¹¹¹In, ¹⁰⁵Rh, ¹⁵³Sm, ⁶⁷Cu, ⁶⁷Ga, ¹⁶⁶Ho, ¹⁷⁷Lu, ¹⁸⁶Re and ¹⁸⁸Re. These isotopes typically produce high-energy α- or β-particles which have a short path length. Such radionuclides kill cells to which they are in close proximity, for example neoplastic cells to which the conjugate has attached or has entered. They have little or no effect on non-localized cells and are essentially non-immunogenic.

Exemplary therapeutic moieties also include cytotoxic agents such as cytostatics (e.g., alkylating agents, DNA synthesis inhibitors, DNA-intercalators or cross-linkers, or DNA-RNA transcription regulators), enzyme inhibitors, gene regulators, cytotoxic nucleosides, tubulin binding agents, hormones and hormone antagonists, anti-angiogenesis agents, and the like.

Exemplary therapeutic moieties also include alkylating agents such as the anthracycline family of drugs (e.g., adriamycin, carminomycin, cyclosporin-A, chloroquine, methopterin, mithramycin, porfiromycin, streptonigrin, anthracenediones, and aziridines). In another embodiment, the chemotherapeutic moiety is a cytostatic agent such as a DNA synthesis inhibitor. Examples of DNA synthesis inhibitors include, but are not limited to, methotrexate and dichloromethotrexate, 3-amino-1,2,4-benzotriazine 1,4-dioxide, aminopterin, cytosine j-D-arabinofuranoside, 5-fluoro-5′-deoxyuridine, 5-fluorouracil, ganciclovir, hydroxyurea, actinomycin-D, and mitomycin C. Exemplary DNA-intercalators or cross-linkers include, but are not limited to, bleomycin, carboplatin, carmustine, chlorambucil, cyclophosphamide, cis-diammineplatinum(II) dichloride (cisplatin), melphalan, mitoxantrone, and oxaliplatin.

Exemplary therapeutic moieties also include transcription regulators such as actinomycin D, daunorubicin, doxorubicin, homoharringtonine, and idarubicin. Other exemplary cytostatic agents that are compatible with the present invention include ansamycin benzoquinones, quinonoid derivatives (e.g., quinolones, genistein, bactacyclin), busulfan, ifosfamide, mechlorethamine, triaziquone, diaziquone, carbazilquinone, indoloquinone EO9, diaziridinyl-benzoquinone methyl DZQ, triethylenephosphoramide, and nitrosourea compounds (e.g., carmustine, lomustine, semustine).

Exemplary therapeutic moieties also include cytotoxic nucleosides such as, for example, adenosine arabinoside, cytarabine, cytosine arabinoside, 5-fluorouracil, fludarabine, floxuridine, ftorafur, and 6-mercaptopurine; tubulin binding agents such as taxoids (e.g., paclitaxel, docetaxel, taxane), nocodazole, rhizoxin, dolastatins (e.g., Dolastatin-10, -11, or -15), colchicine and colchicinoids (e.g., ZD6126), combretastatins (e.g., Combretastatin A-4, AVE-6032), and vinca alkaloids (e.g. vinblastine, vincristine, vindesine, and vinorelbine (navelbine)); anti-angiogenesis compounds such as Angiostatin K1-3, DL-α-difluoromethyl-omithine, endostatin, fumagillin, genistein, minocycline, staurosporine, and (±)-thalidomide.

Exemplary therapeutic moieties also include hormones and hormone antagonists, such as corticosteroids (e.g., prednisone), progestins (e.g., hydroxyprogesterone or medroprogesterone), estrogens, (e.g., diethylstilbestrol), antiestrogens (e.g., tamoxifen), androgens (e.g., testosterone), aromatase inhibitors (e.g., aminogluthetimide), 17-(allylamino)-17-demethoxygeldanamycin, 4-amino-1,8-naphthalimide, apigenin, brefeldin A, cimetidine, dichloromethylene-diphosphonic acid, leuprolide (leuprorelin), luteinizing hormone-releasing hormone, pifithrin-α, rapamycin, sex hormone-binding globulin, and thapsigargin.

Exemplary therapeutic moieties also include enzyme inhibitors such as, S(+)-camptothecin, curcumin, (−)-deguelin, 5,6-dichlorobenz-imidazole 1-β-D-ribofuranoside, etoposide, formestane, fostriecin, hispidin, 2-imino-1-imidazolidineacetic acid (cyclocreatine), mevinolin, trichostatin A, tyrphostin AG 34, and tyrphostin AG 879.

Exemplary therapeutic moieties also include gene regulators such as 5-aza-2′-deoxycytidine, 5-azacytidine, cholecalciferol (vitamin D3), 4-hydroxytamoxifen, melatonin, mifepristone, raloxifene, trans-retinal (vitamin A aldehydes), retinoic acid, vitamin A acid, 9-cis-retinoic acid, 13-cis-retinoic acid, retinol (vitamin A), tamoxifen, and troglitazone.

Exemplary therapeutic moieties also include cytotoxic agents such as, for example, the pteridine family of drugs, diynenes, and the podophyllotoxins. Particularly useful members of those classes include, for example, methopterin, podophyllotoxin, or podophyllotoxin derivatives such as etoposide or etoposide phosphate, leurosidine, vindesine, leurosine and the like.

Still other cytotoxins that are compatible with the teachings herein include auristatins (e.g., auristatin E and monomethylauristan E), calicheamicin, gramicidin D, maytansanoids (e.g., maytansine), neocarzinostatin, topotecan, taxanes, cytochalasin B, ethidium bromide, emetine, tenoposide, colchicin, dihydroxy anthracindione, mitoxantrone, procaine, tetracaine, lidocaine, propranolol, puromycin, and analogs or homologs thereof.

Techniques for conjugating such therapeutic moiety to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

To increase the half-life of the antibodies or polypeptide containing the amino acid sequences described herein, one can attach a salvage receptor binding epitope to the antibody or antigen-binding fragment thereof (especially an antibody fragment), as described, e.g., in U.S. Pat. No. 5,739,277. The term “salvage receptor binding epitope” may refer to an epitope of the Fc region of an IgG molecule (e.g., IgG1, IgG2, IgG3, or IgG4) that is responsible for increasing the in vivo serum half-life of the IgG molecule (e.g., Ghetie et al., 18 Ann. Rev. Immunol. 739 (2000). Antibodies with substitutions in an Fc region thereof and increased serum half-lives are also described in WO 00/42072, WO 02/060919; Shields et al., 276 J. Biol. Chem. 6591 (2001); Hinton, 279 J. Biol. Chem. 6213-6216 (2004). For example, a nucleic acid molecule encoding the salvage receptor binding epitope can be linked in frame to a nucleic acid encoding a polypeptide sequence described herein so that the fusion protein expressed by the engineered nucleic acid molecule comprises the salvage receptor binding epitope and a polypeptide sequence described herein. In another embodiment, the serum half-life can also be increased, for example, by attaching other polypeptide sequences. In one embodiment, the half-life of a Fab is increased by these methods. See also, Dennis et al., 277 J. Biol. Chem. 35035 (2002), for additional serum albumin binding peptide sequences.

Other types of functional moieties are known in the art and can be readily used in the methods and compositions of the present invention based on the teachings contained herein.

### Nucleic Acids

Also provided herein are nucleic acids encoding anti-CD16a antibodies and antigen-binding fragments thereof, as well as vectors, host cells, and expression systems. The term “nucleic acid” as used herein refers to a polymeric form of nucleotides of any length, either ribonucleotides or deoxyribonucleotides. Thus, this term includes, but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases.

The nucleic acids encoding CD16a antibodies and antigen-binding fragments thereof may be, e.g., DNA, cDNA, RNA, synthetically produced DNA or RNA, or a recombinantly produced chimeric nucleic acid molecule comprising any of those polynucleotides either alone or in combination. For example, provided is an expression vector comprising a polynucleotide sequence encoding an anti-CD16a antibody or antigen-binding fragment thereof described herein operably linked to expression control sequences suitable for expression in a eukaryotic and/or prokaryotic host cell.

The term “vector” refers to a vehicle that comprises a polynucleotide to be delivered into a host cell, either in vitro or in vivo. A “vector” includes, but is not limited to, a viral vector, a plasmid, an RNA vector or a linear or circular DNA or RNA molecule which may consists of a chromosomal, non-chromosomal, semi-synthetic or synthetic nucleic acids. In some embodiments, the employed vectors are those capable of autonomous replication (episomal vector) and/or expression of nucleic acids to which they are linked (expression vectors). Large numbers of suitable vectors are known to those of skill in the art and commercially available. Viral vectors include retrovirus, adenovirus, parvovirus (e.g., adeno associated viruses, AAV), coronavirus, negative strand RNA viruses such as orthomyxovirus (e.g., influenza virus), rhabdovirus (e. g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowl pox and canarypox). Other viruses include Norwalk virus, togavirus, flavivirus, reoviruses, papovavirus, hepadnavirus, and hepatitis virus, for example. Examples of retroviruses include avian leukosis-sarcoma, mammalian C-type, B-type viruses, D type viruses, HTLV-BLV group, lentivirus, and spumavirus.

A variety of expression vectors have been developed for the efficient synthesis of antibodies and antigen-binding fragments thereof in prokaryotic cells such as bacteria and in eukaryotic systems, including but not limited to yeast and mammalian cell culture systems have been developed. The vectors can comprise segments of chromosomal, non-chromosomal and synthetic DNA sequences. Also provided are cells comprising expression vectors for the expression of the contemplated CD16a antibodies or antigen-binding fragments thereof.

In one embodiment, provided is a nucleic acid comprising SEQ ID NO:10 and/or 11. In one embodiment, provided is a codon-optimized version of a nucleic acid comprising SEQ ID NO:10 and/or 11. In one embodiment, provided is a nucleic acid comprising SEQ ID NO:13. In one embodiment, provided is a codon-optimized version of a nucleic acid comprising SEQ ID NO: 13.

### Antibody Preparation and Expression Systems

The antibodies or antigen-binding fragments thereof of the invention are typically produced by recombinant expression. Nucleic acids encoding light and heavy chain variable regions, optionally linked to constant regions, are inserted into expression vectors. The light and heavy chains can be cloned in the same or different expression vectors. The DNA segments encoding immunoglobulin chains are operably linked to control sequences in the expression vector(s) that ensure the expression of immunoglobulin polypeptides. Expression control sequences include, but are not limited to, promoters (e.g., naturally-associated or heterologous promoters), signal sequences, enhancer elements, and transcription termination sequences. Preferably, the expression control sequences are eukaryotic promoter systems in vectors capable of transforming or transfecting eukaryotic host cells. Once the vector has been incorporated into the appropriate host, the host is maintained under conditions suitable for high level expression of the nucleotide sequences, and the collection and purification of the cross-reacting antibodies.

These expression vectors are typically replicable in the host organisms either as episomes or as an integral part of the host chromosomal DNA. Commonly, expression vectors contain selection markers (e.g., ampicillin-resistance, hygromycin-resistance, tetracycline resistance or neomycin resistance) to permit detection of those cells transformed with the desired DNA sequences (see, e.g., Itakura et al., U.S. Pat. No. 4,704,362).

The expression of the antibodies and antigen-binding fragments contemplated by the invention can occur in either prokaryotic or eukaryotic cells. Suitable hosts include bacterial or eukaryotic hosts, including yeast, insects, fungi, bird and mammalian cells either in vivo, or in situ, or host cells of mammalian, insect, bird or yeast origin. The mammalian cell or tissue can be of human, primate, hamster, rabbit, rodent, cow, pig, sheep, horse, goat, dog or cat origin, but any other mammalian cell may be used.

E. coli is one prokaryotic host particularly useful for cloning the polynucleotides (e.g., DNA sequences) of the present invention. Other microbial hosts suitable for use include bacilli, such as Bacillus subtillis, and other enterobacteriaceae, such as Salmonella, Serratia, and various Pseudomonas species.

Other microbes, such as yeast, are also useful for expression. Saccharomyces and Pichia are exemplary yeast hosts, with suitable vectors having expression control sequences (e.g., promoters), an origin of replication, termination sequences and the like as desired. Typical promoters include 3-phosphoglycerate kinase and other glycolytic enzymes. Inducible yeast promoters include, among others, promoters from alcohol dehydrogenase, isocytochrome C, and enzymes responsible for methanol, maltose, and galactose utilization.

Further, by use of, for example, the yeast ubiquitin hydrolase system, in vivo synthesis of ubiquitin-transmembrane polypeptide fusion proteins can be accomplished. The fusion proteins so produced can be processed in vivo or purified and processed in vitro, allowing synthesis of an anti-CD16a antibody or antigen-binding fragment thereof of the present invention with a specified amino terminus sequence. Moreover, problems associated with retention of initiation codon-derived methionine residues in direct yeast (or bacterial) expression maybe avoided. Sabin et al., 7 Bio/Technol. 705 (1989); Miller et al., 7 Bio/Technol. 698 (1989).

Any of a series of yeast gene expression systems incorporating promoter and termination elements from the actively expressed genes coding for glycolytic enzymes produced in large quantities when yeast is grown in mediums rich in glucose can be utilized to obtain recombinant CD16a antibodies or peptides of the present invention. Known glycolytic genes can also provide very efficient transcriptional control signals. For example, the promoter and terminator signals of the phosphoglycerate kinase gene can be utilized.

Production of CD16a antibodies or antigen-binding fragments thereof in insects can be achieved, for example, by infecting the insect host with a baculovirus engineered to express a transmembrane polypeptide by methods known to those of skill in the art. See Ausubel et al., 1987, 1993.

In addition to microorganisms, mammalian tissue culture may also be used to express and produce the antibodies or antigen-binding fragments thereof of the present invention (e.g., polynucleotides encoding immunoglobulins or fragments thereof). See Winnacker, From Genes to Clones, VCH Publishers, N.Y., N.Y. (1987). Eukaryotic cells are actually preferred, because a number of suitable host cell lines capable of secreting heterologous proteins (e.g., intact immunoglobulins) have been developed in the art, and include CHO cell lines, various COS cell lines, HeLa cells, 293 cells, myeloma cell lines, transformed B-cells, and hybridomas. Expression vectors for these cells can include expression control sequences, such as an origin of replication, a promoter, and an enhancer (Queen et al., Immunol. Rev. 89:49 (1986)), and necessary processing information sites, such as ribosome binding sites, RNA splice sites, polyadenylation sites, and transcriptional terminator sequences. Preferred expression control sequences are promoters derived from immunoglobulin genes, SV40, adenovirus, bovine papilloma virus, cytomegalovirus and the like. See Co et al., J. Immunol. 148:1149 (1992).

Alternatively, nucleotide sequences encoding antibodies or antigen-binding fragments thereof can be incorporated in transgenes for introduction into the genome of a transgenic animal and subsequent expression in the milk of the transgenic animal (see, e.g., Deboer et al., U.S. Pat. No. 5,741,957, Rosen, U.S. Pat. No. 5,304,489, and Meade et al., U.S. Pat. No. 5,849,992). Suitable transgenes include coding sequences for light and/or heavy chains in operable linkage with a promoter and enhancer from a mammary gland specific gene, such as casein or beta lactoglobulin.

Additionally, plants have emerged as a convenient, safe and economical alternative main-stream expression systems for recombinant antibody production, which are based on large scale culture of microbes or animal cells. Antibodies or antigen-binding fragments thereof can be expressed in plant cell culture, or plants grown conventionally. The expression in plants may be systemic, limited to sub-cellular plastids, or limited to seeds (endosperms). See, e.g., U.S. Patent Pub. No. 2003/0167531; U.S. Pat. Nos. 6,080,560 and 6,512,162; and WO 0129242. Several plant-derived antibodies have reached advanced stages of development, including clinical trials (see, e.g., Biolex, NC).

The vectors containing the polynucleotide sequences of interest (e.g., the heavy and light chain encoding sequences and expression control sequences) can be transferred into the host cell by well-known methods, which vary depending on the type of cellular host. For example, calcium chloride transfection is commonly utilized for prokaryotic cells, whereas calcium phosphate treatment, electroporation, lipofection, biolistics or viral-based transfection may be used for other cellular hosts. (See generally Sambrook et al., Molecular Cloning: A Laboratory Manual (Cold Spring Harbor Press, 2nd ed., 1989). Other methods used to transform mammalian cells include the use of polybrene, protoplast fusion, liposomes, electroporation, and microinjection (see generally, Sambrook et al., supra). For production of transgenic animals, transgenes can be microinjected into fertilized oocytes, or can be incorporated into the genome of embryonic stem cells, and the nuclei of such cells transferred into enucleated oocytes.

The antibodies and antigen-binding fragments thereof of the invention can be expressed using a single vector or two vectors. When the antibody heavy and light chains are cloned on separate expression vectors, the vectors are co-transfected to obtain expression and assembly of intact immunoglobulins. Once expressed, the whole antibodies, their dimers, individual light and heavy chains, or other immunoglobulin forms of the present invention can be purified according to standard procedures of the art, including ammonium sulfate precipitation, affinity columns, column chromatography, HPLC purification, gel electrophoresis and the like (see generally Scopes, Protein Purification (Springer-Verlag, N.Y., (1982)). Substantially pure immunoglobulins of at least about 90 to 95% homogeneity are preferred, and 98 to 99% or more homogeneity most preferred, for pharmaceutical uses.

In embodiments, the fusion protein is expressed with a signal sequence (also referred to as a signal peptide), which mediated translocation of the nascent antibody protein from the cytosol into the ER. Signal peptides frequently contain 5-30 amino acids and are present at the N-terminus of the nascent antibody protein. Signal sequences suitable for antibody production are well known in the art. See, e.g., Haryadi et al. Optimization of heavy chain and light chain signal peptides for high level expression of therapeutic antibodies in CHO cells. PLoS One. 2015 Feb. 23; 10(2):e0116878; Ling et al. Essentially Leading Antibody Production: An Investigation of Amino Acids, Myeloma, and Natural V-Region Signal Peptides in Producing Pertuzumab and Trastuzumab Variants, Front Immunol. 2020 Dec. 7; 11:604318, which are incorporated herein in their entireties. In some embodiments, the anti-CD16 antibody or antibody fragment is expressed with a signal sequence comprising a sequence that is at least at least 80%, at least 85%, at least 90%, at least 95%, at least 96%, at least 97%, at least 98%, or at least 99% identical to SEQ ID NO:14. In one embodiment, the anti-CD16 antibody or antibody fragment is expressed with a signal sequence comprising SEQ ID NO:14.

In one embodiment, the heavy and the light chain of the anti-CD16 antibody or antigen-binding fragment thereof is expressed as a fusion protein. Provided herein is a fusion protein comprising (1) an anti-CD16 antibody heavy variable chain, and (2) an anti-CD16 antibody light variable chain. Provided herein is a fusion protein comprising (1) a signal peptide, (2) an anti-CD16 antibody heavy variable chain, and (3) an anti-CD16 antibody light variable chain. Provided herein is a fusion protein comprising (1) a signal peptide, (2) an anti-CD16 antibody heavy variable chain, (3) a cleavage site, and (4) an anti-CD16 antibody light variable chain.

Provided herein is a fusion protein comprising (1) an anti-CD16 antibody heavy variable region, (2) an anti-CD16 antibody heavy constant region, (3) an anti-CD16 antibody light variable region, (4) an anti-CD16 antibody light constant region. Provided herein is a fusion protein comprising (1) a signal peptide, (2) an anti-CD16 antibody heavy variable region, (3) an anti-CD16 antibody heavy constant region, (4) an anti-CD16 antibody light variable region, (5) an anti-CD16 antibody light constant region. Provided herein is a fusion protein comprising (1) a signal peptide, (2) an anti-CD16 antibody heavy variable region, (3) an anti-CD16 antibody heavy constant region, (4) an anti-CD16 antibody light variable region, (5) an anti-CD16 antibody light constant region. Provided herein is a fusion protein comprising (1) a signal peptide, (2) an anti-CD16 antibody heavy variable region, (3) an anti-CD16 antibody heavy constant region, (4) a cleavage site, (6) an anti-CD16 antibody light variable region, (6) an anti-CD16 antibody light constant region. In embodiments, the heavy variable region is located N-terminal of the light variable region. In embodiments, the heavy variable region is located C-terminal of the light variable region.

In some embodiments, the cleavage site is a self-cleaving peptide sequence. Self-cleaving peptide sequences are known in the art. See., e.g., Liu et al. Systematic comparison of 2A peptides for cloning multi-genes in a polycistronic vector. Sci Rep. 2017 May 19; 7(1):2193. In one embodiment, the self-cleaving peptide sequence is T2A.

In one embodiment, the fusion protein is at least at least 80%, at least 85%, at least 90%, at least 95%, at least 96%, at least 97%, at least 98%, or at least 99% identical to SEQ ID NO:13 (see Table 3 for annotation). Provided herein is a fusion protein comprising SEQ ID NO:13.

Provided herein is a method of producing an antibody or antigen-binding fragment thereof disclosed, the method comprising providing a cell expressing an antibody or antigen-binding fragment thereof disclosed herein and isolating the antibody or antigen-binding fragment.

### Methods of Using the Anti-CD16a Antibodies and Antigen-Binding Fragments Thereof Described Herein

Provided herein are methods of using the anti-CD16a antibodies and antigen-binding fragments thereof described herein for reducing CD16a shedding by immune cells, for promoting NK cell-driven immunity, and for treating subjects in need thereof.

In one embodiment, the anti-CD16a antibody or antigen-binding fragment thereof is administered in combination with another antibody or antigen-binding fragment that targets a cancer antigen (herein referred to as a “anti-cancer antibody or antigen-binding fragment thereof”). As used herein, a “cancer antigen” or “tumor antigen” is an antigenic substance that is produced in cancer cells. Cancer antigens are typically expressed exclusively expressed by cancer cells or are preferentially expressed on cancer cells as compared to healthy cells.

In some embodiments, the anti-cancer antibody or antigen-binding fragment thereof binds to a cancer antigen selected from the group consisting of epidermal growth factor receptor (EGFR), CD20, HER-2, CD38, Programmed death-ligand 1 (PD-L1) or GD2. Other, non-limiting examples of cancer antigens include CD19, CD123, CD47, CD30, CD33, CD133, BCMA, TEM8, EpCAM, ROR1, Folate Receptor, CD70, MAGE-1, MAGE-2, MAGE-3, MAGE A-10, MAGE-C2, MAGE-A12, CEA, tyrosinase, midkin, BAGE, CASP-8, β-catenin, CA-125, CDK-1, ESO-1, gp75, gp100, MART-1, MUC-1, MUM-1, p53, PAP, PSA, PSMA, ras, trp-1, TRP-1, TRP-2, IL13Ralpha, IL13Ralpha2, AIM-2, AIM-3, NY-ESO-1, C9orf112, SART1, SART2, SART3, BRAP, RTN4, GLEA2, TNKS2, KIAA0376, ING4, HSPH1, C13orf24, RBPSUH, C6orf153, NKTR, NSEP1, U2AF1L, CYNL2, TPR GOLGA, BMI1, COX-2, EGFRvIII, EZH2, LICAM, Livin, Livinβ, MRP-3, Nestin, OLIG2, ART1, ART4, B-cycline, Gli1, Cav-1, Cathepsin B, CD74, E-Cadherin, EphA2/Eck, Fra-1/Fosl 1, GAGE-1, Ganglioside, GnT-V, β1, 6-N, Ki67, Ku70/80, PROX1, PSCA, SOX10, SOX11, Survivin, βhCG, WT1, mesothelin, melan-A, NY-BR-1, NY-CO-58, MN (gp250), telomerase, SSX-2, PRAME, PLK1, VEGF-A, VEGFR2, and Tie-2.

In some embodiments, the anti-cancer antibody is an antibody that binds tumor cells and has a human IgG1 Fc domain. In some embodiments, the anti-cancer antibody is cetuximab, rituximab, trastuzumab, avelumab, necitumumab, daratumumab, dinutuximab, zalutumumab, or nimotuzumab (see Table 1).

TABLE 1
Nonlimiting examples of anti-cancer antibodies that can be in combination
with an anti-CD16 antibody or antigen-binding fragment thereof.

| | | | | Antibody |
|---|---|---|---|---|
| | Example cell | Therapeutic | Antibody | constant |
| Disease | line | antibody | target | region |
| EGFR⁺ non-small | NCI-H1703, | Cetuximab | Epidermal | IgG1 |
| cell lung cancer | NCI-H661 | | growth factor | |
| (NSCLC) | | | receptor | |
| | | | (EGFR) | |
| Epidermoid | A431 | Cetuximab | EGFR | IgG1 |
| carcinoma | | | | |
| EGFR⁺ colorectal | SW480, Caco-2 | Cetuximab | EGFR | IgG1 |
| carcinoma (CRC) | | | | |
| Head and neck | | Cetuximab | EGFR | IgG1 |
| cancer | | | | |
| B-cell lymphoma | Raji, SU-DHL- | Rituximab | CD20 | IgG1 |
| | 6, OCI-Ly10 | | | |
| HER2⁺ breast cancer | MDA-MB543, | Trastuzumab | HER-2 | IgG1 |
| | SKBR3 | | | |
| Gastrointestinal | | Trastuzumab | HER-2 | IgG1 |
| cancers | | | | |
| PD-L1⁺ melanoma | A375, A2058 | Avelumab | Programmed | IgG1 |
| | | | death-ligand 1 | |
| | | | (PD-L1) | |
| Merkel cell and | | Avelumab | PD-L1 | IgG1 |
| urothelial carcinomas | | | | |
| Squamous cell | | Necitumumab | EGFR | IgG1 |
| carcinoma of the | | | | |
| lungs | | | | |
| Multiple myeloma | | Daratumumab | CD38 | IgG1 |
| Neuroblastoma | | Dinutuximab | GD2 | IgG1 |
| Head and neck | | Zalutumumab | EGFR | IgG1 |
| cancer | | | | |
| Non-small cell % | | Zalutumumab | EGFR | IgG1 |
| lung cancer | | | | |
| (NSCLC) | | | | |
| Glioma | | Nimotuzumab | EGFR | IgG1 |
| Head and neck | | Nimotuzumab | EGFR | IgG1 |
| cancer | | | | |
| Non-small cell % | | Nimotuzumab | EGFR | IgG1 |
| lung cancer | | | | |
| (NSCLC) | | | | |

In some embodiments, the anti-cancer antibody or antigen-binding fragment thereof binds to a checkpoint inhibitor. Illustrative checkpoint molecules that may be targeted for binding by the anti-cancer antibody or antigen-binding fragment thereof include, but are not limited to, CTLA-4, PD-L1, PD-L2, PD-1, B7-H3, B7-H4, BTLA, HVEM, GAL9, LAG3, TIM-3, VISTA, KIR, 2B4 (belongs to the CD2 family of molecules and is expressed on all NK, γδ, and memory CD8⁺ (αβ) T cells), CD160 (also referred to as BY55), CGEN-15049, CHK1 and CHK2 kinases, A2aR and various B-7 family ligands. B7 family ligands include, but are not limited to, B7-1, B7-2, B7-DC, B7-H1, B7-H2, B7-H3, B7-H4, B7-H5, B7-H6 and B7-H7. In some embodiments, the anti-cancer antibody or antigen-binding fragment thereof binds to a checkpoint protein ligands including, but are not limited to, PD-L1, PD-L2, B7-H3, B7-H4, CD28, CD86 and TIM-3.

A “combination comprising an anti-CD16a antibody or antigen-binding fragment thereof and an anti-cancer antibody or antigen-binding fragment thereof” as used herein does not require that the anti-CD16a antibody or antigen-binding fragment thereof and the anti-cancer antibody or antigen-binding fragment thereof are provided in the same pharmaceutical composition and/or are formulated together. In embodiments, the anti-CD16a antibody or antigen-binding fragment thereof and the anti-cancer antibody or antigen-binding fragment thereof are provided in different pharmaceutical compositions and/or are formulated together. In embodiments, the anti-CD16a antibody or antigen-binding fragment thereof and the anti-cancer antibody or antigen-binding fragment thereof are provided in the same pharmaceutical compositions and/or are formulated together. In embodiments, the anti-CD16a antibody or antigen-binding fragment thereof is provided concurrently with the anti-cancer antibody or antigen-binding fragment thereof. In embodiments, the anti-CD16a antibody or antigen-binding fragment thereof is provided consecutively with the anti-cancer antibody or antigen-binding fragment thereof. In embodiments, the anti-CD16a antibody or antigen-binding fragment thereof is provided before administration of the anti-cancer antibody or antigen-binding fragment thereof. In embodiments, the anti-CD16a antibody or antigen-binding fragment thereof is provided after the anti-cancer antibody or antigen-binding fragment thereof is provided. In embodiments, the anti-CD16a antibody or antigen-binding fragment thereof is provided before and after the anti-cancer antibody or antigen-binding fragment thereof is provided.

As used herein, the term “CD16a shedding” refers proteolysis of the CD16a ectodomain that involves the membrane-associated protease ADAM17 (a disintegrin and metalloproteinase-17), which is expressed on NK cells and other immune cells (granulocytes, macrophages, and subsets of monocytes, eosinophils, T-cells and dendritic cells). The metalloprotease ADAM17 cleaves the stalk region of CD16a between Ala195 and Val196 proximal to the plasma membrane. Provided herein are anti-CD16 antibodies or antigen-binding fragments that reduce CD16a cleavage. By “reducing” is meant the ability to cause an overall decrease of about 20% or greater, 30% or greater, 40% or greater, 45% or greater, 50% or greater, of 55% or greater, of 60% or greater, of 65% or greater, of 70% or greater, or 75%, 80%, 85%, 90%, 95%, or greater, as compared to a control that is not treated with an anti-CD16a antibody or antigen-binding fragment thereof or that is not treated with an anti-CD16a antibody or antigen-binding fragment thereof and an anti-cancer antibody or antigen-binding fragment thereof. Methods of measuring CD16a shedding are known in the art. By way of non-limiting example, CD16a shedding can be measured using primary NK cells that were treated with PMA to induce the shedding. CD16a shedding can be determined, for example, by detecting soluble CD16a shed by NK cells (e.g., by ELISA) or by detecting remaining surface CD16a on NK cells (e.g., by flow cytometry). Other methods of detecting CD16a shedding/CD16a cleavage are known to the person skilled in the art.

In one aspect, the anti-CD16 antibody or antigen-binding fragment is useful for reducing CD16a shedding. As such, provided herein is a method of reducing CD16a shedding, the method comprising administering to a subject in need thereof an anti-CD16 antibody or antigen-binding fragment.

Further provided herein is a method of reducing CD16a shedding, the method comprising contacting a cell expressing CD16a with an anti-CD16 antibody or antigen-binding fragment. In some embodiments, the cell expressing CD16a is a NK cell. In some embodiments, the cell expressing CD16a is a macrophage.

As used herein, “reducing CD16a shedding” refers a reduction in CD16a shedding upon exposure to an anti-CD16 antibody or antigen-binding fragment thereof disclosed herein (or upon administering to a subject an anti-CD16 antibody or antigen-binding fragment disclosed herein) as compared to a control. In embodiments, the control is a control sample (or a collection of control samples) or control subject (or a population of control subjects) that is not exposed to an anti-CD16 antibody or antigen-binding fragment thereof disclosed herein. In embodiments, the control is a control sample (or a collection of control samples) or control subject (or a population of control subjects) that is exposed to a different anti-CD16 antibody or antigen-binding fragment thereof or a different therapeutic agent or combination of different therapeutic agents. In some embodiments, the reduction is a reduction by at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 95%, or 100%.

In one aspect, the (1) anti-CD16 antibody or antigen-binding fragment or the (2) combination of an anti-CD16 antibody or antigen-binding fragment thereof and an anti-cancer antibody or antigen-binding fragment thereof is useful for increasing NK cell-driven immunity by promoting NK cell effector functions. Methods of measuring NK cell effector functions are known in the art. For example, an increase in NK cell effector function leads to an increase in NK cell degranulation, which can for example, be analyzed by measuring CD107a externalization. CD107a is a transmembrane protein inside granules that contain perforin and granzymes, and it is the traditional marker of NK cell cytotoxicity. Another non-limiting example of measuring NK cell effector function is determining the release of cytokine secreted by the NK cells, including, but not limited to, interferon 7. Other methods of measuring NK cell effector function are known to the person skilled in the art.

Provided herein is a method of increasing NK cell-driven immunity, the method comprising administering to a subject in need thereof an anti-CD16 antibody or antigen-binding fragment. Also provided herein is a method of increasing NK cell-driven immunity, the method comprising administering to a subject in need thereof an anti-CD16 antibody or antigen-binding fragment thereof and an anti-cancer antibody or antigen-binding fragment thereof.

Provided herein is a method of increasing NK cell effector function in a subject in need thereof, the method comprising administering to the subject an anti-CD16 antibody or antigen-binding fragment. Also provided herein is a method of increasing NK cell effector function in a subject in need thereof, the method comprising administering to the subject an anti-CD16 antibody or antigen-binding fragment thereof and an anti-cancer antibody or antigen-binding fragment thereof.

Provided herein is a method of increasing NK cell effector function, the method comprising contacting an NK cell expressing CD16a with an anti-CD16 antibody or antigen-binding fragment. Also provided herein is a method of increasing NK cell effector function, the method comprising contacting an NK cell expressing CD16a with an anti-CD16 antibody or antigen-binding fragment thereof and an anti-cancer antibody or antigen-binding fragment thereof. In some embodiments, the cell expressing CD16a is a NK cell. In some embodiments, the cell expressing CD16a is a macrophage.

Provided herein is a method of increasing NK cell-mediated killing of cancer cells, the method comprising administering to a subject in need thereof an anti-CD16 antibody or antigen-binding fragment. Also provided herein is a method of increasing NK cell-mediated killing of cancer cells, the method comprising administering to a subject in need thereof an anti-CD16 antibody or antigen-binding fragment thereof and an anti-cancer antibody or antigen-binding fragment thereof.

Provided herein is a method of increasing NK cell-mediated killing of cancer cells, the method comprising contacting an NK cell expressing CD16a with an anti-CD16 antibody or antigen-binding fragment. Also provided herein is a method of increasing NK cell-mediated killing of cancer cells, the method comprising contacting an NK cell expressing CD16a with an anti-CD16 antibody or antigen-binding fragment thereof and an anti-cancer antibody or antigen-binding fragment thereof.

Provided herein is a method of increasing NK cell-mediated ADCC in a subject in need thereof, the method comprising administering to the subject an anti-CD16 antibody or antigen-binding fragment. Also provided herein is a method of increasing NK cell-mediated ADCC in a subject in need thereof, the method comprising administering to the subject an anti-CD16 antibody or antigen-binding fragment thereof and an anti-cancer antibody or antigen-binding fragment thereof.

Provided herein is a method of increasing NK cell-mediated ADCC in a subject in need thereof, the method comprising contacting an NK cell expressing CD16a with an anti-CD16 antibody or antigen-binding fragment. Also provided herein is a method of increasing NK cell-mediated ADCC in a subject in need thereof, the method comprising contacting an NK cell expressing CD16a with an anti-CD16 antibody or antigen-binding fragment thereof and an anti-cancer antibody or antigen-binding fragment thereof.

As used herein, “increasing NK cell-driven immunity” refers an increase in NK cell-driven immunity upon administering to a subject of (1) an anti-CD16 antibody or antigen-binding fragment thereof disclosed herein or (2) a combination of an anti-CD16 antibody or antigen-binding fragment thereof disclosed herein and an anti-cancer antibody or antigen-binding fragment thereof as compared to a control. In embodiments, the control is a control subject (or a population of control subjects) that is not exposed to (1) an anti-CD16 antibody or antigen-binding fragment thereof disclosed herein or (2) a combination of an anti-CD16 antibody or antigen-binding fragment thereof disclosed herein and an anti-cancer antibody or antigen-binding fragment thereof. In embodiments, the control is a control subject (or a population of control subjects) that is exposed to (1) a different anti-CD16 antibody or antigen-binding fragment thereof, (2) a different therapeutic agent, or (3) a combination of different therapeutic agents. In some embodiments, the increase is an increase by at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 95%, or 100%.

As used herein, “increasing NK cell effector function,” “increasing NK cell-mediated killing of cancer cells,” and “increasing NK cell-mediated ADCC” refers to an increase in NK cell effector function, NK cell-mediated killing of cancer cells, or NK cell-mediated ADCC, respectively, upon exposure to (or upon administering to a subject of) (1) an anti-CD16 antibody or antigen-binding fragment thereof disclosed herein or (2) a combination of an anti-CD16 antibody or antigen-binding fragment thereof disclosed herein and an anti-cancer antibody or antigen-binding fragment thereof as compared to a control. In embodiments, the control is a control sample (or a collection of control samples) or a control subject (or a population of control subjects) that is not exposed to (1) an anti-CD16 antibody or antigen-binding fragment thereof disclosed herein or (2) a combination of an anti-CD16 antibody or antigen-binding fragment thereof disclosed herein and an anti-cancer antibody or antigen-binding fragment thereof. In embodiments, the control is a control sample (or a collection of control samples) or a control subject (or a population of control subjects) that is exposed to (1) a different anti-CD16 antibody or antigen-binding fragment thereof, (2) a different therapeutic agent, or (3) a combination of different therapeutic agents. In some embodiments, the increase is an increase by at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 95%, or 100%.

In some embodiments, the (1) anti-CD16 antibody or antigen-binding fragment or the (2) combination of an anti-CD16 antibody or antigen-binding fragment thereof and an anti-cancer antibody or antigen-binding fragment thereof promotes ADCC by immune cells expressing CD16a other than NK cells, such as granulocytes, macrophages, and subsets of monocytes, eosinophils, T-cells and dendritic cells.

In one aspect, provided is an (1) anti-CD16 antibody or antigen-binding fragment or a (2) combination of an anti-CD16 antibody or antigen-binding fragment thereof and an anti-cancer antibody or antigen-binding fragment thereof that are useful for the treatment of subjects in need thereof.

In the methods described herein, a therapeutically effective amount of an antibody or antigen-binding portions thereof set forth herein is administered to a mammal in need thereof. Although antibodies or antigen-binding portions thereof set forth herein are particularly useful for administration to humans, they may be administered to other mammals as well. The term “mammal” as used herein is intended to include, but is not limited to, humans, laboratory animals, domestic pets and farm animals. “Therapeutically effective amount” means an amount of antibody or antigen-binding portions thereof set forth herein that, when administered to a mammal, is effective in producing the desired therapeutic effect.

Provided herein are methods of treating a subject having a cancer or tumor and methods of reducing tumor growth, comprising administering an effective amount of an anti-CD16a antibody or antigen-binding fragment thereof provided herein. “Reducing” includes inhibiting and/or reversing and can refer to, for example, the symptoms of the disorder being treated, the presence or size of metastases or micrometastases, the size of the primary tumor, the presence or the size of the dormant tumor.

Provided herein is a method of treating cancer in a subject in need thereof, the method comprising administering to the subject an anti-CD16a antibody or antigen-binding fragment thereof provided herein. Provided herein is a method of treating cancer in a subject in need thereof, the method comprising administering to the subject an anti-CD16a antibody or antigen-binding fragment thereof provided herein and an anti-cancer antibody or antigen-binding fragment thereof. Provided herein is the use of an anti-CD16a antibody or antigen-binding fragment thereof provided herein for treating cancer in a subject in need thereof. Provided herein is the use of a combination of an anti-CD16a antibody or antigen-binding fragment thereof provided herein and an anti-cancer antibody or antigen-binding fragment thereof for treating cancer in a subject in need thereof. Provided herein is the use of an anti-CD16a antibody or antigen-binding fragment thereof provided herein in the manufacture of a medicament or in the manufacture of a pharmaceutical composition for treating cancer in a subject in need thereof. Provided herein is the use of a combination of an anti-CD16a antibody or antigen-binding fragment thereof provided herein and an anti-cancer antibody or antigen-binding fragment thereof in the manufacture of a medicament or in the manufacture of a pharmaceutical composition for treating cancer in a subject in need thereof.

Provided herein is a method of reducing tumor growth in a subject in need thereof, the method comprising administering to the subject an anti-CD16a antibody or antigen-binding fragment thereof provided herein. Provided herein is a method of reducing tumor growth in a subject in need thereof, the method comprising administering to the subject an anti-CD16a antibody or antigen-binding fragment thereof provided herein and an anti-cancer antibody or antigen-binding fragment thereof. Provided herein is the use of an anti-CD16a antibody or antigen-binding fragment thereof provided herein for reducing tumor growth in a subject in need thereof. Provided herein is the use of a combination of an anti-CD16a antibody or antigen-binding fragment thereof provided herein and an anti-cancer antibody or antigen-binding fragment thereof for reducing tumor growth in a subject in need thereof. Provided herein is the use of an anti-CD16a antibody or antigen-binding fragment thereof provided herein in the manufacture of a medicament or in the manufacture of a pharmaceutical composition for reducing tumor growth in a subject in need thereof. Provided herein is the use of a combination of an anti-CD16a antibody or antigen-binding fragment thereof provided herein and an anti-cancer antibody or antigen-binding fragment thereof in the manufacture of a medicament or in the manufacture of a pharmaceutical composition for reducing tumor growth in a subject in need thereof.

The term “cancer” refers to or describes the physiological condition in mammals that is typically characterized by unregulated cell growth. Included in this definition are benign and malignant cancers, as well as dormant tumors or micrometastases. Accordingly, the term “cancer” as used herein refers to an uncontrolled growth of cells, which interferes with the normal functioning of the bodily organs and systems, including cancer stem cells and tumor vascular niches. A subject that has a cancer is a subject having objectively measurable cancer cells present in the subject's body. Included in this definition are benign and malignant cancers, as well as dormant tumors or micrometastases. Cancers that migrate from their original location and seed vital organs can eventually lead to the death of the subject through the functional deterioration of the affected organs. Hematopoietic cancers, such as leukemia, are able to out-compete the normal hematopoietic compartments in a subject, thereby leading to hematopoietic failure (in the form of anemia, thrombocytopenia and neutropenia) ultimately causing death.

By “subject” is meant a mammal, including, but not limited to, a human or non-human mammal, such as a bovine, equine, canine, ovine, or feline, etc. Individuals and patients are also subjects herein.

The terms “treat,” “treated,” “treating,” or “treatment” as used herein refer to therapeutic treatment, wherein the object is to slow down (lessen) an undesired physiological condition, disorder or disease, or to obtain beneficial or desired clinical results. For the purposes of this invention, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. The terms “prevent”, “prevention”, and the like refer to acting prior to overt disease or disorder onset, to prevent the disease or disorder from developing or to minimize the extent of the disease or disorder or slow its course of development.

The embodiments of the invention may be used for treating cancer metastasis, which relates to the spreading of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass. Both stimulatory and inhibitory molecular pathways within the tumor cell regulate this behavior, and interactions between the tumor cell and host cells in the distant site are also significant. Metastases are most often detected through the sole or combined use of magnetic resonance imaging (MRI) scans, computed tomography (CT) scans, blood and platelet counts, liver function studies, chest X-rays and bone scans in addition to the monitoring of specific symptoms.

Provided herein is a method of treating cancer metastasis in a subject in need thereof, the method comprising administering to the subject an anti-CD16a antibody or antigen-binding fragment thereof provided herein. Provided herein is a method of treating cancer metastasis in a subject in need thereof, the method comprising administering to the subject an anti-CD16a antibody or antigen-binding fragment thereof provided herein and an anti-cancer antibody or antigen-binding fragment thereof. Provided herein is the use of an anti-CD16a antibody or antigen-binding fragment thereof provided herein for treating cancer metastasis in a subject in need thereof. Provided herein is the use of a combination of an anti-CD16a antibody or antigen-binding fragment thereof provided herein and an anti-cancer antibody or antigen-binding fragment thereof for treating cancer metastasis in a subject in need thereof. Provided herein is the use of an anti-CD16a antibody or antigen-binding fragment thereof provided herein in the manufacture of a medicament or in the manufacture of a pharmaceutical composition for treating cancer metastasis in a subject in need thereof. Provided herein is the use of a combination of an anti-CD16a antibody or antigen-binding fragment thereof provided herein and an anti-cancer antibody or antigen-binding fragment thereof in the manufacture of a medicament or in the manufacture of a pharmaceutical composition for treating cancer metastasis in a subject in need thereof.

Also contemplated are methods of reducing cancer stemness comprising the administration of the anti-CD16a antibodies or antigen-binding fragments thereof disclosed herein. Cancer sternness may refer to the ability of a cell to self-renew and to generate an additional, phenotypically distinct cell type. Cancer stem cells (CSCs) are cancer cells that exhibit stem-cell like properties. CSCs often exhibit at least one hallmark of cancer, and is capable of generating at least one additional, phenotypically distinct cell type. Furthermore, cancer stem cells are capable of both asymmetric and symmetric replication. It is appreciated that a cancer stem cell may result from differentiated cancer cells that acquire sternness traits and/or stem cells that acquire phenotypes associated with cancer cells. Alternatively, cancer stem cells can reconstitute non-stromal cell types within a tumor.

Provided herein is a method of reducing cancer stemness in a subject in need thereof, the method comprising administering to the subject an anti-CD16a antibody or antigen-binding fragment thereof provided herein. Provided herein is a method of reducing cancer stemness in a subject in need thereof, the method comprising administering to the subject an anti-CD16a antibody or antigen-binding fragment thereof provided herein and an anti-cancer antibody or antigen-binding fragment thereof. Provided herein is the use of an anti-CD16a antibody or antigen-binding fragment thereof provided herein for reducing cancer stemness in a subject in need thereof. Provided herein is the use of a combination of an anti-CD16a antibody or antigen-binding fragment thereof provided herein and an anti-cancer antibody or antigen-binding fragment thereof for reducing cancer stemness in a subject in need thereof. Provided herein is the use of an anti-CD16a antibody or antigen-binding fragment thereof provided herein in the manufacture of a medicament or in the manufacture of a pharmaceutical composition for reducing cancer sternness in a subject in need thereof. Provided herein is the use of a combination of an anti-CD16a antibody or antigen-binding fragment thereof provided herein and an anti-cancer antibody or antigen-binding fragment thereof in the manufacture of a medicament or in the manufacture of a pharmaceutical composition for reducing cancer sternness in a subject in need thereof.

In a further embodiment, provided is a method of decreasing tumor-associated fibrosis, the method comprising administering to a subject in need thereof an anti-CD16a antibody or antigen-binding fragment herein.

Provided herein is a method of decreasing tumor-associated fibrosis in a subject in need thereof, the method comprising administering to the subject an anti-CD16a antibody or antigen-binding fragment thereof provided herein. Provided herein is a method of decreasing tumor-associated fibrosis in a subject in need thereof, the method comprising administering to the subject an anti-CD16a antibody or antigen-binding fragment thereof provided herein and an anti-cancer antibody or antigen-binding fragment thereof. Provided herein is the use of an anti-CD16a antibody or antigen-binding fragment thereof provided herein for decreasing tumor-associated fibrosis in a subject in need thereof. Provided herein is the use of a combination of an anti-CD16a antibody or antigen-binding fragment thereof provided herein and an anti-cancer antibody or antigen-binding fragment thereof for decreasing tumor-associated fibrosis in a subject in need thereof. Provided herein is the use of an anti-CD16a antibody or antigen-binding fragment thereof provided herein in the manufacture of a medicament or in the manufacture of a pharmaceutical composition for decreasing tumor-associated fibrosis in a subject in need thereof. Provided herein is the use of a combination of an anti-CD16a antibody or antigen-binding fragment thereof provided herein and an anti-cancer antibody or antigen-binding fragment thereof in the manufacture of a medicament or in the manufacture of a pharmaceutical composition for decreasing tumor-associated fibrosis in a subject in need thereof.

Cancers that may be treated by the compositions and methods contemplated by the invention include tumors that are not vascularized, or not yet substantially vascularized, as well as vascularized tumors. The cancers may comprise nonsolid tumors (such as hematological tumors, for example, leukemias and lymphomas) or may comprise solid tumors. Types of cancers to be treated include, but are not limited to benign and malignant tumors, and malignancies e.g., sarcomas, carcinomas, and melanomas. Adult tumors/cancers and pediatric tumors/cancers are also included. Examples of cancer include but are not limited to, carcinoma, lymphoma, blastoma, sarcoma, and leukemia. More particular examples of such cancers include, but are not limited to, basal cell carcinoma, biliary tract cancer; bladder cancer; bone cancer; brain and CNS cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); lymphoma including Hodgkin's and non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; vulvar sarcomas; as well as other carcinomas and sarcomas; as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome. A patient can have more than one type of cancer.

The efficacy of the treatment methods for cancer comprising therapeutic formulations of the compositions comprising the antibodies and antigen-binding fragments thereof described herein can be measured by various endpoints commonly used in evaluating cancer treatments, including but not limited to, tumor regression, tumor weight or size shrinkage, time to progression, duration of survival, progression free survival, overall response rate, duration of response, and quality of life. In the case of cancers, the therapeutically effective amount of the recombinant anti-CD16a antibody or antigen-binding fragment thereof can reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. In cases where a patient has more than one type of cancer, the therapeutically effective amount of the recombinant anti-CD16a antibody or antigen-binding fragment thereof is an amount effective in treating at least one of the cancers. To the extent the recombinant anti-CD16a antibody or antigen binding-fragment thereof acts to prevent growth and/or kill existing cancer cells, it can be cytostatic and/or cytotoxic. For cancer therapy, efficacy in vivo can, for example, be measured by assessing the duration of survival, duration of progression free survival (PFS), the response rates (RR), duration of response, and/or quality of life.

### Pharmaceutical Compositions

In another aspect, the present invention provides pharmaceutically acceptable compositions that comprise a therapeutically effective amount of (1) an anti-CD16a antibody or antigen-binding fragment thereof, (2) an anti-cancer antibody or antigen-binding fragment thereof, or (3) a combination of an anti-CD16a antibody or antigen-binding fragment thereof and anti-cancer antibody or antigen-binding fragment thereof, formulated together with one or more pharmaceutically acceptable excipients.

The dosage of active agent(s) may vary, depending on the reason for use, the individual subject, and the mode of administration. The dosage may be adjusted based on the subject's weight, the age and health of the subject, and tolerance for the compound(s) or composition. For example, depending on the disease, for (1) an anti-CD16a antibody or antigen-binding fragment thereof, (2) an anti-cancer antibody or antigen-binding fragment thereof, or (3) a combination of an anti-CD16a antibody or antigen-binding fragment thereof and anti-cancer antibody or antigen-binding fragment thereof, this may require 0.1, 1.0, 3.0, 6.0, or 10.0 mg/kg. For an IgG having a molecular mass of 150,000 g/mole (two binding sites), these doses correspond to approximately 18 nM, 180 nM, 540 nM, 1.08 μM, and 1.8 μM of binding sites for a 5 L blood volume.

The active agent and excipient(s) may be formulated into compositions and dosage forms according to methods known in the art. The pharmaceutical compositions of the present invention may be specially formulated in solid or liquid form, including those adapted for parenteral administration, for example, by subcutaneous, intratumoral, intramuscular or intravenous injection as, for example, a sterile solution or suspension.

Therapeutic compositions comprising (1) an anti-CD16a antibody or antigen-binding fragment thereof, (2) an anti-cancer antibody or antigen-binding fragment thereof, or (3) a combination of an anti-CD16a antibody or antigen-binding fragment thereof and anti-cancer antibody or antigen-binding fragment thereof may formulated with one or more pharmaceutically-acceptable excipients, which can be a pharmaceutically-acceptable material, composition or vehicle, such as a liquid or solid filler, diluent, carrier, manufacturing aid (e.g., lubricant, talc magnesium, calcium or zinc stearate, or steric acid), solvent or encapsulating material, involved in carrying or transporting the therapeutic compound for administration to the subject, bulking agent, salt, surfactant and/or a preservative. Some examples of materials which can serve as pharmaceutically-acceptable excipients include: sugars, such as lactose, glucose and sucrose; starches, such as corn starch and potato starch; cellulose and its derivatives, such as sodium carboxymethyl cellulose, ethyl cellulose and cellulose acetate; gelatin; talc; waxes; oils, such as peanut oil, cottonseed oil, safflower oil, sesame oil, olive oil, corn oil and soybean oil; glycols, such as ethylene glycol and propylene glycol; polyols, such as glycerin, sorbitol, mannitol and polyethylene glycol; esters, such as ethyl oleate and ethyl laurate; agar; buffering agents; water; isotonic saline; pH buffered solutions; and other non-toxic compatible substances employed in pharmaceutical formulations.

A bulking agent is a compound which adds mass to a pharmaceutical formulation and contributes to the physical structure of the formulation in lyophilized form. Suitable bulking agents according to the present invention include mannitol, glycine, polyethylene glycol and sorbitol.

The use of a surfactant can reduce aggregation of the reconstituted protein and/or reduce the formation of particulates in the reconstituted formulation. The amount of surfactant added is such that it reduces aggregation of the reconstituted protein and minimizes the formation of particulates after reconstitution. Suitable surfactants according to the present invention include polysorbates (e.g., polysorbates 20 or 80); poloxamers (e.g., poloxamer 188); Triton; sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g., lauroamidopropyl); myristamidopropyl-, palmidopropyl-, or isostearamidopropyl-dimethylamine; sodium methyl cocoyl-, or disodium methyl oleyl-taurate; and polyethyl glycol, polypropyl glycol, and copolymers of ethylene and propylene glycol (e.g., Pluronics, PF68, etc.).

Preservatives may be used in formulations of invention. Suitable preservatives for use in the formulation of the invention include octadecyldimethylbenzyl ammonium chloride, hexamethonium chloride, benzalkonium chloride (a mixture of alkylbenzyl-dimethylammonium chlorides in which the alkyl groups are long-chain compounds), and benzethonium chloride. Other types of preservatives include aromatic alcohols such as phenol, butyl and benzyl alcohol, alkyl parabens such as methyl or propyl paraben, catechol, resorcinol, cyclohexanol, 3-pentanol, and m-cresol. Other suitable excipients can be found in standard pharmaceutical texts, e.g., in “Remington's Pharmaceutical Sciences”, The Science and Practice of Pharmacy, 19th Ed. Mack Publishing Company, Easton, Pa., (1995).

The compositions comprising (1) an anti-CD16a antibody or antigen-binding fragment thereof, (2) an anti-cancer antibody or antigen-binding fragment thereof, or (3) a combination of an anti-CD16a antibody or antigen-binding fragment thereof and anti-cancer antibody or antigen-binding fragment thereof and a pharmaceutically acceptable carrier may comprise the antibody or antigen-binding fragment thereof (or antibodies and antigen-binding fragments thereof) at various concentrations. For example, the compositions may comprise an antibody or antigen-binding fragment (or antibodies and antigen-binding fragments thereof) thereof at 10 mg/ml to 200 mg/ml, 25 mg/ml to 130 mg/ml, 50 mg/ml to 125 mg/ml, 75 mg/ml to 110 mg/ml, or 80 mg/ml to 100 mg/ml. The compositions also may comprise an antibody or antigen-binding fragment thereof at about 10 mg/ml, 20 mg/ml, 30 mg/ml, 40 mg/ml, 50 mg/ml, 60 mg/ml, 70 mg/ml, 80 mg/ml, 90 mg/ml, 100 mg/ml, 110 mg/ml, 120 mg/ml, 130 mg/ml, 140 mg/ml, or 150 mg/ml.

In some embodiments, the compositions comprising (1) the anti-CD16a antibody or antigen-binding fragment thereof, (2) the anti-cancer antibody or antigen-binding fragment thereof, or (3) the combination of an anti-CD16a antibody or antigen-binding fragment thereof and anti-cancer antibody or antigen-binding fragment thereof and the pharmaceutically acceptable carrier are lyophilized and provided in a composition for reconstitution prior to administration.

### Methods of Administration

Therapeutic compositions comprising the (1) the anti-CD16a antibody or antigen-binding fragment thereof, (2) the anti-cancer antibody or antigen-binding fragment thereof, or (3) the combination of an anti-CD16a antibody or antigen-binding fragment thereof and anti-cancer antibody or antigen-binding fragment thereof may be administered in any convenient manner, including by injection, transfusion, implantation or transplantation. The compositions described herein may be administered to a patient subcutaneously, intradermally, intratumorally, intranodally, intramedullary, intramuscularly, intracranially, by intravenous or intralymphatic injection, or intraperitoneally. In one embodiment, the cell compositions of the present invention are preferably administered by intravenous injection.

In certain embodiments, (1) the anti-CD16a antibody or antigen-binding fragment thereof, (2) the anti-cancer antibody or antigen-binding fragment thereof, or (3) the combination of an anti-CD16a antibody or antigen-binding fragment thereof and anti-cancer antibody or antigen-binding fragment thereof is administered to the mammal by intravenous infusion, i.e., introduction of (1) the anti-CD16a antibody or antigen-binding fragment thereof, (2) the anti-cancer antibody or antigen-binding fragment thereof, or (3) the combination of an anti-CD16a antibody or antigen-binding fragment thereof and anti-cancer antibody or antigen-binding fragment thereof into the vein of a mammal over a certain period of time. In certain embodiments, the period of time is about 5 minutes, about 10 minutes, about 30 minutes, about 1 hour, about 2 hours, about 4 hours, or about 8 hours.

In certain embodiments, a dose of a compound or a composition is administered to a subject every day, every other day, every couple of days, every third day, once a week, twice a week, three times a week, once every two weeks, or once a month. In other embodiments, two, three or four doses of a compound or a composition is administered to a subject every day, every couple of days, every third day, once a week, once every two weeks or once a month. In some embodiments, a dose(s) of a compound or a composition is administered for 2 days, 3 days, 5 days, 7 days, 14 days, 21 days or 28 days. In certain embodiments, a dose of a compound or a composition is administered for 1 month, 1.5 months, 2 months, 2.5 months, 3 months, 4 months, 5 months, 6 months or more.

### Combination Therapies

Provided herein are (1) an anti-CD16a antibody or antigen-binding fragment thereof, (2) an anti-cancer antibody or antigen-binding fragment thereof, or (3) a combination of an anti-CD16a antibody or antigen-binding fragment thereof and anti-cancer antibody or antigen-binding fragment thereof that is administered with one or more additional therapeutic agents. Such additional agents include, but are not limited to, cytotoxic agents, chemotherapeutic agents, growth inhibitory agents, anti-inflammatory agents, anti-cancer agents, anti-neurodegenerative agents, and anti-infective agents. Agents that are used in such combination therapies may fall into one or more of the preceding categories. The administration of (1) the anti-CD16a antibody or antigen-binding fragment thereof, (2) the anti-cancer antibody or antigen-binding fragment thereof, or (3) the combination of an anti-CD16a antibody or antigen-binding fragment thereof and anti-cancer antibody or antigen-binding fragment thereof and the additional therapeutic agent may be concurrently or consecutively. The administration of (1) the anti-CD16a antibody or antigen-binding fragment thereof, (2) the anti-cancer antibody or antigen-binding fragment thereof, or (3) the combination of an anti-CD16a antibody or antigen-binding fragment thereof and anti-cancer antibody or antigen-binding fragment thereof and the additional therapeutic agent may be separately or as a mixture. Further, the methods of treatment contemplated by the invention can relate to a treatment in combination with one or more cancer therapies selected from the group of antibody therapy, chemotherapy, cytokine therapy, dendritic cell therapy, gene therapy, hormone therapy, laser light therapy, and radiation therapy.

Exemplary additional therapeutic agents also include radionuclides with high-energy ionizing radiation that are capable of causing multiple strand breaks in nuclear DNA, and therefore suitable for inducing cell death (e.g., of a cancer). Exemplary high-energy radionuclides include: ⁹⁰Y, ¹²⁵I, ¹³¹I, ¹²³I, ¹¹¹In, ¹⁰⁵Rh, ¹⁵³Sm, ⁶⁷Cu, ⁶⁷Ga, ¹⁶⁶Ho, ¹⁷⁷Lu, ¹⁸⁶Re and ¹⁸⁸Re. These isotopes typically produce high energy α- or β-particles which have a short path length. Such radionuclides kill cells to which they are in close proximity, for example neoplastic cells to which the conjugate has attached or has entered. They have little or no effect on non-localized cells and are essentially non-immunogenic.

Exemplary additional therapeutic agents also include cytotoxic agents such as cytostatics (e.g., alkylating agents, DNA synthesis inhibitors, DNA-intercalators or cross-linkers, or DNA-RNA transcription regulators), enzyme inhibitors, gene regulators, cytotoxic nucleosides, tubulin binding agents, hormones and hormone antagonists, anti-angiogenesis agents, and the like.

Exemplary additional therapeutic agents also include alkylating agents such as the anthracycline family of drugs (e.g., adriamycin, carminomycin, cyclosporin-A, chloroquine, methopterin, mithramycin, porfiromycin, streptonigrin, anthracenediones, and aziridines). In another embodiment, the chemotherapeutic moiety is a cytostatic agent such as a DNA synthesis inhibitor. Examples of DNA synthesis inhibitors include, but are not limited to, methotrexate and dichloromethotrexate, 3-amino-1,2,4-benzotriazine 1,4-dioxide, aminopterin, cytosine β-D-arabinofuranoside, 5-fluoro-5′-deoxyuridine, 5-fluorouracil, ganciclovir, hydroxyurea, actinomycin-D, and mitomycin C. Exemplary DNA-intercalators or cross-linkers include, but are not limited to, bleomycin, carboplatin, carmustine, chlorambucil, cyclophosphamide, cis-diammineplatinum(II) dichloride (cisplatin), melphalan, mitoxantrone, and oxaliplatin.

Exemplary additional therapeutic agents also include transcription regulators such as actinomycin D, daunorubicin, doxorubicin, homoharringtonine, and idarubicin. Other exemplary cytostatic agents that are compatible with the present invention include ansamycin benzoquinones, quinonoid derivatives (e.g., quinolones, genistein, bactacyclin), busulfan, ifosfamide, mechlorethamine, triaziquone, diaziquone, carbazilquinone, indoloquinone EO9, diaziridinyl-benzoquinone methyl DZQ, triethylenephosphoramide, and nitrosourea compounds (e.g., carmustine, lomustine, semustine).

Exemplary additional therapeutic agents also include cytotoxic nucleosides such as, for example, adenosine arabinoside, cytarabine, cytosine arabinoside, 5-fluorouracil, fludarabine, floxuridine, ftorafur, and 6-mercaptopurine; tubulin binding agents such as taxoids (e.g. paclitaxel, docetaxel, taxane), nocodazole, rhizoxin, dolastatins (e.g., Dolastatin-10, -11, or -15), colchicine and colchicinoids (e.g., ZD6126), combretastatins (e.g., Combretastatin A-4, AVE-6032), and vinca alkaloids (e.g., vinblastine, vincristine, vindesine, and vinorelbine (navelbine)); anti-angiogenesis compounds such as Angiostatin K1-3, DL-α-difluoromethyl-ornithine, endostatin, fumagillin, genistein, minocycline, staurosporine, and (±)-thalidomide.

Exemplary additional therapeutic agents also include hormones and hormone antagonists, such as corticosteroids (e.g., prednisone), progestins (e.g., hydroxyprogesterone or medroprogesterone), estrogens, (e.g., diethylstilbestrol), antiestrogens (e.g., tamoxifen), androgens (e.g., testosterone), aromatase inhibitors (e.g., aminogluthetimide), 17-(allylamino)-17-demethoxygeldanamycin, 4-amino-1,8-naphthalimide, apigenin, brefeldin A, cimetidine, dichloromethylene-diphosphonic acid, leuprolide (leuprorelin), luteinizing hormone-releasing hormone, pifithrin-α, rapamycin, sex hormone-binding globulin, and thapsigargin.

Exemplary additional therapeutic agents also include enzyme inhibitors such as, S(+)-camptothecin, curcumin, (−)-deguelin, 5,6-dichlorobenz-imidazole 1-β-D-ribofuranoside, etoposide, formestane, fostriecin, hispidin, 2-imino-1-imidazolidineacetic acid (cyclocreatine), mevinolin, trichostatin A, tyrphostin AG 34, and tyrphostin AG 879.

Exemplary additional therapeutic agents also include gene regulators such as 5-aza-2′-deoxycytidine, 5-azacytidine, cholecalciferol (vitamin D3), 4-hydroxytamoxifen, melatonin, mifepristone, raloxifene, trans-retinal (vitamin A aldehydes), retinoic acid, vitamin A acid, 9-cis-retinoic acid, 13-cis-retinoic acid, retinol (vitamin A), tamoxifen, and troglitazone.

Exemplary additional therapeutic agents also include cytotoxic agents such as, for example, the pteridine family of drugs, diynenes, and the podophyllotoxins. Particularly useful members of those classes include, for example, methopterin, podophyllotoxin, or podophyllotoxin derivatives such as etoposide or etoposide phosphate, leurosidine, vindesine, leurosine and the like.

Still other additional therapeutic agents that are compatible with the teachings herein include auristatins (e.g., auristatin E and monomethylauristan E), calicheamicin, gramicidin D, maytansanoids (e.g., maytansine), neocarzinostatin, topotecan, taxanes, cytochalasin B, ethidium bromide, emetine, tenoposide, colchicin, dihydroxy anthracindione, mitoxantrone, procaine, tetracaine, lidocaine, propranolol, puromycin, and analogs or homologs thereof.

In some embodiments, the additional therapeutic agent is a checkpoint inhibitor. The checkpoint inhibitor may be an antibody or antigen-binding fragment thereof, a binding protein, a biologic or a small molecule.

### Kits

Also provided herein are kits comprising (1) an anti-CD16 antibody or antigen-binding fragment thereof (or conjugate thereof) or (2) a combination of an anti-CD16 antibody or antigen-binding fragment thereof (or conjugate thereof) and an anti-cancer antibody or antigen-binding fragment thereof (or conjugate thereof) described herein. In embodiments, the kit comprises the antibody or antibodies (or antigen-binding fragment thereof or conjugate thereof) in lyophilized form in a first container, and an optional second container comprising sterile water, sterile buffered water, or at least one preservative selected from the group consisting of phenol, m-cresol, p-cresol, o-cresol, chlorocresol, benzyl alcohol, phenylmercuric nitrite, phenoxyethanol, formaldehyde, chlorobutanol, magnesium chloride, alkylparaben, benzalkonium chloride, benzethonium chloride, sodium dehydroacetate and thimerosal, or mixtures thereof in an aqueous diluent.

Also provided is an article of manufacture for human pharmaceutical use, comprising packaging material and a container comprising (1) an anti-CD16 antibody or antigen-binding fragment thereof (or conjugate thereof) or (2) a combination of an anti-CD16 antibody or antigen-binding fragment thereof (or conjugate thereof) and an anti-cancer antibody or antigen-binding fragment thereof (or conjugate thereof) described herein. The article of manufacture can optionally comprise having the container as a component of a parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, or transdermal delivery device or system.

It is to be understood that this invention is not limited to the particular molecules, compositions, methodologies, or protocols described, as these may vary. Any methods and materials similar or equivalent to those described herein can be used in the practice or testing of embodiments of the present invention. It is further to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes those possibilities).

All references, patents and applications cited herein are incorporated herein by reference in their entireties. Furthermore, where a definition or use of a term in a reference, which is incorporated by reference herein is inconsistent or contrary to the definition of that term provided herein, the definition of that term provided herein applies and the definition of that term in the reference does not apply.

To facilitate a better understanding of the present invention, the following examples of specific embodiments are given. The following examples should not be read to limit or define the entire scope of the invention.

### EXAMPLES

### Example 1: Generation of Anti-CD16a Antibodies

To generate anti-CD16a antibodies capable of inhibiting CD16a shredding from cells, a hybridoma approach was employed. Supernatants were obtained from hybridomas that secrete anti-human CD16a antibodies. Primary natural killer (NK) cells were treated with phorbol myristate acetate (PMA) to rapidly induce CD16a downregulation by proteolytic cleavage. The cells were incubated with the hybridoma supernatants, and anti-CD16a antibody binding to the NK cells was determined using an ELISA. To exclude antibody clones that block the Fc recognition site of CD16a, phycoerythrin (PE)-labelled CD16a antibody 3G8 (known to block CD16a by binding to the Fc recognition site of CD16a) was used to stain CD16a on NK cells.

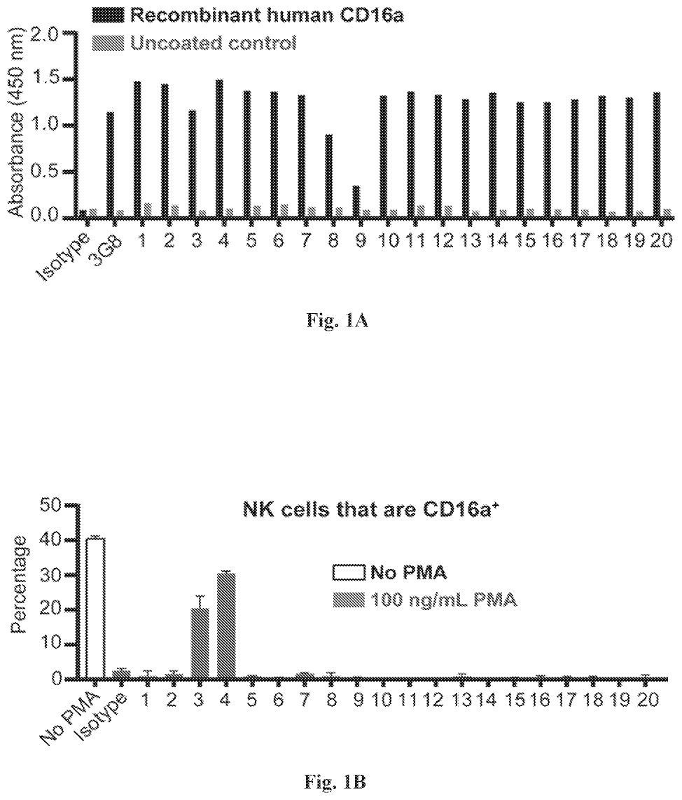

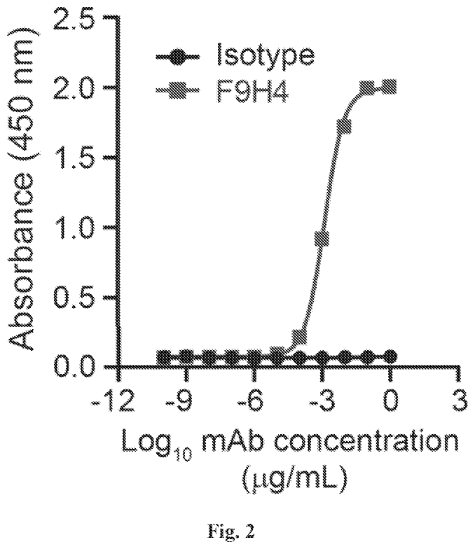

Only hybridomas 3 (“F9”) and 4 (“H4”) were found to produce anti-CD16 antibodies that inhibit the CD16a shedding by PMA-treated NK cells (FIG. 1). Hybridoma sequencing revealed that both hybridomas encoded the same anti-CD16a antibody, called F9H4 herein. The sequence information for anti-CD16a antibody F9H4 is shown in Table 2. Binding of F9H4 to human CD16a protein was demonstrated by ELISA (FIG. 2).

TABLE 2
CDR, heavy and variable regions, and heavy and variable
constant regions of anti-hCD16a antibody F9H4.

| Name | Amino acid sequence | DNA sequence |
|---|---|---|
| CDR1H | SEQ ID NO: 1 | |
| CDR2H | SEQ ID NO: 2 | |
| CDR3H | SEQ ID NO: 3 | |
| CDR1L | SEQ ID NO: 4 | |
| CDR2L | RTS | |
| CDR3L | SEQ ID NO: 5 | |
| V_{H} | SEQ ID NO: 6 | SEQ ID NO: 10 |
| V_{L} | SEQ ID NO: 8 | SEQ ID NO: 11 |
| HC with DANA mutation | SEQ ID NO: 7 | |
| HC without DANA mutation | SEQ ID NO: 15 | |
| LC | SEQ ID NO: 9 | |
| HC/LC fusion protein | SEQ ID NO: 12 | SEQ ID NO: 13 |

### Example 2: Anti-CD16a Antibody F9H4 Inhibits CD16a Cleavage

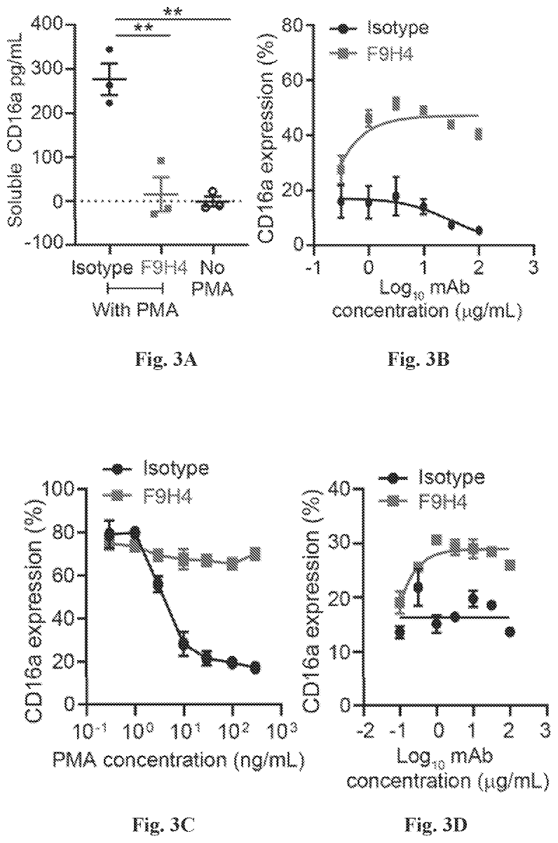

To demonstrate that F9H4 inhibits CD16a cleavage, primary NK cells were isolated from peripheral blood of healthy donor. The surface proteins of the NK cells were covalently linked to biotin. The cells were treated for four hours with protein kinase C agonist (PMA) to induce cleavage. The supernatants were collected and biotinylated CD16a captured in an ELISA plate using anti-CD16a antibody 3G8 (a commercially available anti-CD16a monoclonal antibody that does not compete with F9H4). Biotinylated CD16a was detected with peroxidase-labelled streptavidin. F9H4 completely stopped the shedding of CD16a into supernatants of PMA-treated NK cells (FIG. 3A). As consequence, F9H4 retained CD16a on the cellular surface, as determined by labelling NK cells with PE-conjugated 3G8 and followed by flow cytometry (FIG. 3B). F9H4 did not increase expression of CD16a, but rather retained CD16a on the surface of PMA-treated NK cells (FIG. 3C). Similar results were obtained with monocyte-derived macrophages (FIG. 3D).

In sum, F9H4 inhibits CD16a shedding by NK cells and macrophages.

### Example 3: Characterization of Anti-CD16a Antibody F9H4

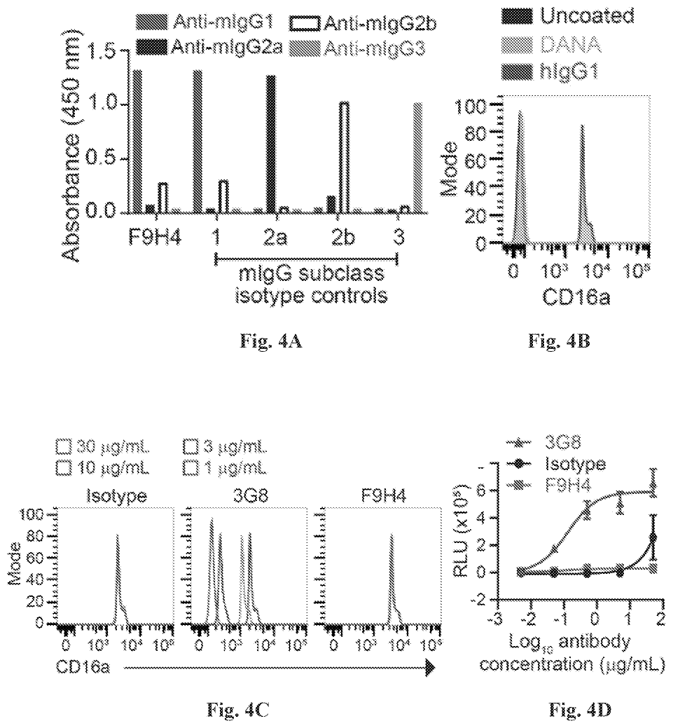

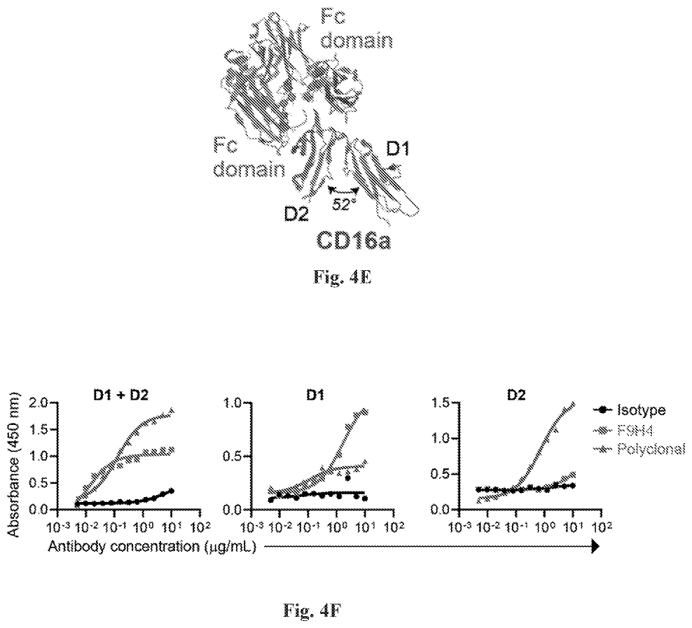

F9H4 was shown to be an mIgG1 antibody, which is a heavy chain with Fc domain known not to bind with high affinity to human Fc activating receptors and, as consequence, it cannot cause the bonafide cell depletion (FIG. 4A). That feature enables the in vitro and in vivo testing of the parental version of F9H4. As demonstrated using a flow-cytometry-based bead assay that analyzes the binding between human IgG1 (hIgG1) and CD16a (FIG. 4B), F9H4 did not obstruct interaction between CD16a and the Fc domain of hIgG1 (FIG. 4C). In contrast, control anti-CD16a antibody 3G8 inhibited the binding in a dose-dependent manner (FIG. 4C) because the antibody's epitope is located in the CD16a's Fc domain recognition site. Using an engineered cell line that serves as CD16a engagement reporter, it was shown that 3G8 was agonistic because it mimicked Fc domain binding. In contrast, F9H4 was found to be non-agonistic (FIG. 4D).

To shed light into the mechanism by which F9H4 inhibit the cleavage, the two immunoglobulin-like domains, D1 and D2 of CD16a were examined. D2 binds the Fc domain, whereas D1 does not participate in that interaction (FIG. 4E). Since F9H4 does not block CD16a (FIG. 4C), it was hypothesized that F9H4 binds D1. Three recombinant proteins were generated that consist of full length CD16a (D1+D2), D1 domain, and D2 domain. It was determined by ELISA that F9H4 binds full length CD16a and D1, but not D2. A polyclonal antibody that binds all three recombinant proteins (FIG. 4F) was used as a positive control. Therefore, F9H4 is a D1-specific antibody.

In sum, F9H4 does not cause CD16a blockade or engagement. Further, F9H4 is unlikely to induce cell depletion because it is an mIgG1 antibody.

### Example 4: Anti-CD16a Antibody F9H4 Promotes NK Cell-Mediated ADCC In Vitro

Next, it was demonstrated that F9H4 promotes NK cell-mediated ADCC of tumor cells that are tagged with antibodies against surface proteins, wherein the antibodies comprise hIgG1 heavy chains for CD16a engagement. To that end, a human lung adenocarcinoma cell line (A549) tagged with cetuximab was used as a “target cell”. Cetuximab is an anti-epidermal growth factor receptor (EGFR) antibody. Cell line A549 expresses EGFR wild type. As NK cells isolated from healthy donors were used as effector cells. NK cells were co-cultured with A549 cells in the presence of F9H4 or an isotype control antibody, followed by flow cytometry analyses.

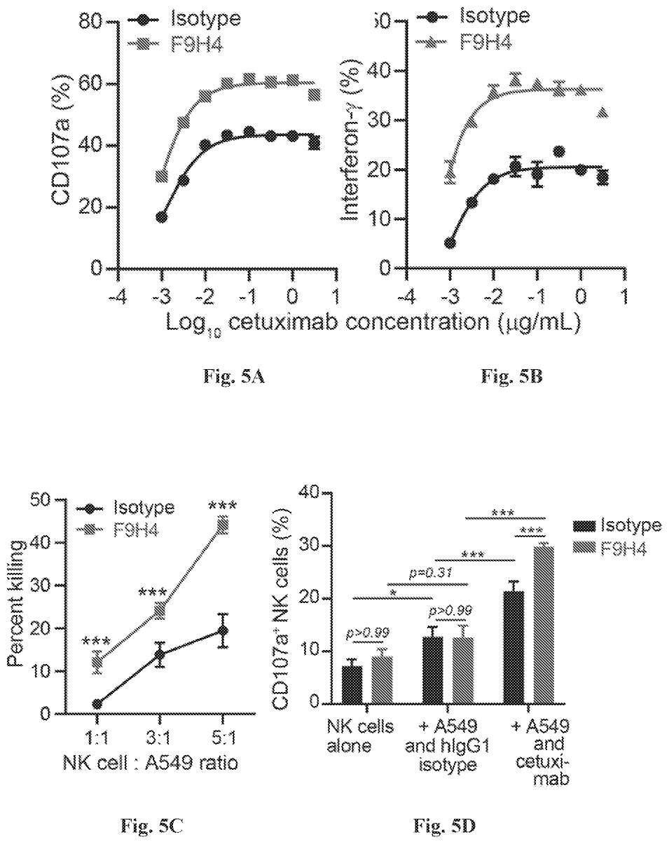

The major effector functions of NK cells were analyzed after four-to-six-hour co-cultures. F9H4 increases the percentage of NK cell degranulation against cetuximab-tagged A549 cells, as analyzed by CD107a externalization (FIG. 5A). CD107a is a transmembrane protein inside granules that contain perforin and granzymes, and it is the traditional marker of NK cell cytotoxicity against target cells. F9H4 also increases production of interferon γ by NK cells co-cultured with cetuximab-tagged A549 (FIG. 5B). As consequence, F9H4 increases the NK cell-mediated killing of cetuximab-tagged A549 cells (FIG. 5C). F9H4 does not trigger NK cell degranulation in the absence of CD16a engagement by cetuximab-tagged A549, as determined in mechanistic experiment with additional control groups (FIG. 5D). A549 induces CD16a downregulation by NK cells that is promoted by cetuximab, whereas F9H4 significantly inhibited the downregulation in both conditions (FIG. 5E).

In sum, F9H4 promotes NK cell-mediated ADCC by inhibiting CD16a cleavage, which occurs upon contact with tumor cells and cetuximab.

### Example 5: a Combination of Anti-CD16a Antibody F9H4 and Anti-EGFR Antibody Cetuximab Promotes NK Cell-Mediated Anti-Tumor Immunity and Inhibit Human Lung Cancer In Vivo

Transition to in vivo experiments is challenging because the Fe receptor biology differs substantially between mice and humans in terms of receptor structure, expression partner, and profile of binding to Fc domains. For example, mice have only one isoform of CD16 (in contrast to humans, which have two isoforms, CD16a and CD16b). The extracellular domain of murine CD16 has only 49% amino acid identity with human CD16a. For those reasons, wild type mice are not suitable for assessing NK cell-mediated anti-tumor immunity. On the other hand, hIL-15 NOG mice are immunodeficient and constitutively express human IL-15 to enable long-term engraftment of human NK cells.

hIL-15 NOG mice were inoculated intravenously with human NK cells that had been isolated from healthy donors. Two weeks later, it was confirmed by flow cytometry that the mice were successfully engrafted with human NK cells. Mice in a separate cohort were also inoculated intravenously with A549, and three weeks later it was found by histopathology that A549 forms tumors in the lungs. Therefore, A549 was used as a human lung cancer model to study the in vivo activity of anti-CD16 antibody F9H4 and cetuximab in hIL-15 NOG mice reconstituted with human NK cells.

New cohorts of hIL-15 NOG mice were inoculated with human NK cells from healthy donors. One week later, the mice were inoculated with A549 cells. Treatments with F9H4, cetuximab, and/or isotype controls started one day later. Analyses of metastases by histopathology were performed on day 21.

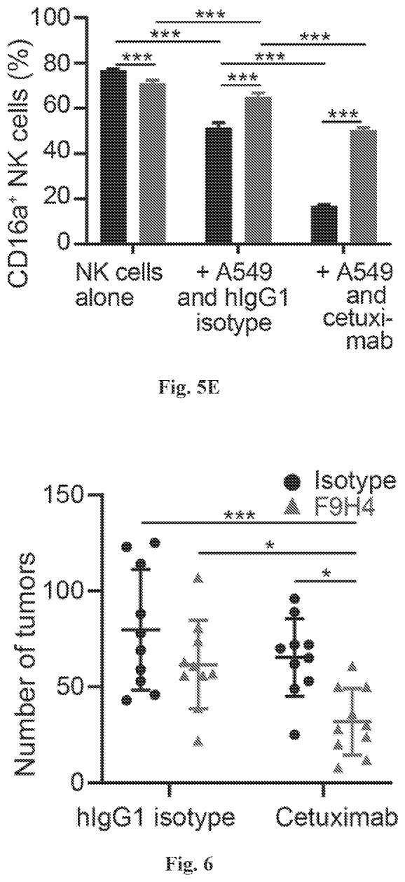

A combination of F9H4 and cetuximab significantly inhibited the formation of tumors in the lungs, compared to all other treatments groups (FIG. 6).

In sum, F9H4 and cetuximab promote NK cell-mediated anti-tumor immunity and inhibit human lung cancer in vivo.

### Example 6: Development of Chimeric Version of Anti-CD16 Antibody F9H4 with an Fc Domain Mutation

F9H4 is a murine antibody directed against a human protein. To generate a chimeric version of murine antibody F9H14, the antibody variable regions of F9H4 were cloned into a vector that enables expression of a recombinant, chimeric antibody comprising the murine variable regions of F9H4, the CH1 region of F9H4, and a human hIG1 Fc domain region (comprising CD2 and CH3) in CHO cells. The resulting construct is called chF9H4 herein. Additionally, D265A N297A (DANA) mutations were introduced into hIG1, mutations which are known to eliminate binding between Fc domain and Fe receptor. The resulting construct is called chF8H4-DANA herein.

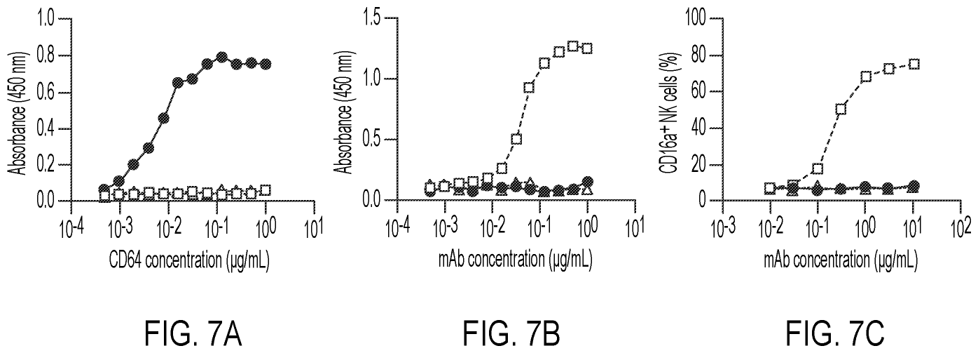

To test whether the DANA mutation successfully abrogated Fc receptor binding, the binding of chF8H4-DANA to CD64 was determined. As expected, chF8H4-DANA did not bind human Fc receptor CD64 (FIG. 7A). Of note, CD16a would not be appropriate for such validation because F9H4 binds to CD16a through the antibody's variable regions.

Further, since the DANA mutations are located in the Fc domain, chF8H4-DANA maintains binding to CD16a and still inhibits the shedding by NK cells (FIGS. 7B and 7C).

TABLE 3
Overview of sequences.

| SEQ ID | | | |
|---|---|---|---|
| NO | Name | Comment | Sequence |
| 1 | CDR1H | | GYTFTSYW |
| 2 | CDR2H | | IYPGSGST |
| 3 | CDR3H | | TMRYGGYYGYYFDY |
| 4 | CDR1L | | SSISSNY |
| | CDR2L | | RTS |
| 5 | CDR3L | | QQGSSIPLT |
| 6 | V_{H} | CDRs | QVQLQQPGSELVRPGASVKLSCKASGYTFTSYWM |
| | | | HWVKQRHGQGLEWIGNIYPGSGSTNYDEKFKSKG |
| | | | TLTVDTSSSTAYMHLGSLTSEDSAVYYCTMRYGG |
| | | | YYGYYFDYWGQGTTLTVSS |
| 7 | HC | V_{H} | QVQLQQPGSELVRPGASVKLSCKASGYTFTSYWM |
| | | D265A N297A | HWVKQRHGQGLEWIGNIYPGSGSTNYDEKFKSKG |
| | | (DANA | TLTVDTSSSTAYMHLGSLTSEDSAVYYCTMRYGG |
| | | mutation) | YYGYYFDYWGQGTTLTVSSAKTTAPSVYPLAPVC |
| | | | GDTTGSSVTLGCLVKGYFPEPVTLTWNSGSLSSG |
| | | | VHTFPAVLQSDLYTLSSSVTVTSSTWPSQSITCN |
| | | | VAHPASSTKVDKKIEPRGPTIKPCPPCKCPAPEL |
| | | | LGGPSVFLFPPKPKDTLMISRTPEVTCVVVAVSH |
| | | | EDPEVKFNWYVDGVEVHNAKTKPREEQYASTYRV |
| | | | VSVLTVLHQDWLNGKEYKCKVSNKALPAPIEKTI |
| | | | SKAKGQPREPQVYTLPPSRDELTKNQVSLTCLVK |
| | | | GFYPSDIAVEWESNGQPENNYKTTPPVLDSDGSF |
| | | | FLYSKLTVDKSRWQQGNVFSCSVMHEALHNHYTQ |
| | | | KSLSLSPGK |
| 8 | V_{L} | CDRs | EIVLTQSPTTMAASPGEKITITCSTTSSISSNYL |
| | | | HWYQQKPGFSPKLLIYRTSNLASGVPARFSGSGS |
| | | | GTSYSLTIGTMEAEDVATYYCQQGSSIPLTFGAG |
| | | | TKLELKR |
| 9 | LC (κ) | V_{L} | EIVLTQSPTTMAASPGEKITITCSTTSSISSNYL |
| | | | HWYQQKPGFSPKLLIYRTSNLASGVPARFSGSGS |
| | | | GTSYSLTIGTMEAEDVATYYCQQGSSIPLTFGAG |
| | | | TKLELKRADAAPTVSIFPPSSEQLTSGGASVVCF |
| | | | LNNFYPKDINVKWKIDGSERQNGVLNSWTDQDSK |
| | | | DSTYSMSSTLTLTKDEYERHNSYTCEATHKTSTS |
| | | | PIVKSFNRNEC |
| 10 | V_{H} | | CAGGTCCAACTGCAGCAACCTGGGTCTGAGCTGG |
| | | | TGAGGCCTGGAGCTTCAGTGAAGCTGTCCTGCAA |
| | | | GGCTTCTGGCTACACATTCACCAGCTACTGGATG |
| | | | CACTGGGTGAAGCAGAGGCATGGACAAGGCCTTG |
| | | | AGTGGATTGGAAATATTTATCCTGGTAGTGGTAG |
| | | | TACTAACTACGATGAGAAGTTCAAGAGCAAGGGC |
| | | | ACACTGACTGTAGACACATCCTCCAGCACAGCCT |
| | | | ACATGCACCTCGGCAGCCTGACATCTGAGGACTC |
| | | | TGCGGTCTATTACTGTACAATGCGCTACGGTGGT |
| | | | TACTACGGGTACTACTTTGACTACTGGGGCCAAG |
| | | | GCACCACTCTCACAGTCTCCTCA |
| 11 | V_{L} | | GAAATTGTGCTCACCCAGTCTCCAACCACCATGG |
| | | | CTGCATCTCCCGGGGAGAAGATCACTATCACCTG |
| | | | CAGTACCACCTCAAGTATAAGTTCCAATTACTTG |
| | | | CATTGGTATCAGCAGAAGCCAGGATTCTCCCCTA |
| | | | AACTCTTGATTTATAGGACATCCAATCTGGCTTC |
| | | | TGGAGTCCCAGCTCGCTTCAGTGGCAGTGGGTCT |
| | | | GGGACCTCTTACTCTCTCACAATTGGCACCATGG |
| | | | AGGCTGAAGATGTTGCCACTTACTACTGCCAGCA |
| | | | GGGTAGTAGTATACCGCTCACGTTCGGTGCTGGG |
| | | | ACCAAGCTGGAGCTGAAACGG |
| 12 | Fusion | Signal peptide | MVPCTLLLLLAAALAPTQTRAQVQLQQPGSELVR |
| | protein | V_{H} | PGASVKLSCKASGYTFTSYWMHWVKQRHGQGLEW |
| | | Self-cleaving | IGNIYPGSGSTNYDEKFKSKGTLTVDTSSSTAYM |
| | | T2A peptide | HLGSLTSEDSAVYYCTMRYGGYYGYYFDYWGQGT |
| | | sequence | TLTVSSAKTTAPSVYPLAPVCGDTTGSSVTLGCL |
| | | V_{L} | VKGYFPEPVTLTWNSGSLSSGVHTFPAVLQSDLY |
| | | D265A N297A | TLSSSVTVTSSTWPSQSITCNVAHPASSTKVDKK |
| | | (DANA | IEPRGPTIKPCPPCKCPAPELLGGPSVFLFPPKP |
| | | mutation) | KDTLMISRTPEVTCVVVAVSHEDPEVKFNWYVDG |
| | | | VEVHNAKTKPREEQYASTYRVVSVLTVLHQDWLN |
| | | | GKEYKCKVSNKALPAPIEKTISKAKGQPREPQVY |
| | | | TLPPSRDELTKNQVSLTCLVKGFYPSDIAVEWES |
| | | | NGQPENNYKTTPPVLDSDGSFFLYSKLTVDKSRW |
| | | | QQGNVFSCSVMHEALHNHYTQKSLSLSPGKEGRG |
| | | | SLLTCGDVEENPGPMVPCTLLLLLAAALAPTQTR |
| | | | AEIVLTQSPTTMAASPGEKITITCSTTSSISSNY |
| | | | LHWYQQKPGFSPKLLIYRTSNLASGVPARFSGSG |
| | | | SGTSYSLTIGTMEAEDVATYYCQQGSSIPLTFGA |
| | | | GTKLELKRADAAPTVSIFPPSSEQLTSGGASVVC |
| | | | FLNNFYPKDINVKWKIDGSERQNGVLNSWTDQDS |
| | | | KDSTYSMSSTLTLTKDEYERHNSYTCEATHKTST |
| | | | SPIVKSFNRNEC |
| 13 | Fusion | Signal peptide | ATGGTACCGTGCACGCTGCTCCTGCTGTTGGCGG |
| | protein | Self-cleaving | CCGCCCTGGCTCCGACTCAGACCCGCGCGCAGGT |
| | | T2A peptide | CCAACTGCAGCAACCTGGGTCTGAGCTGGTGAGG |
| | | sequence | CCTGGAGCTTCAGTGAAGCTGTCCTGCAAGGCTT |
| | | | CTGGCTACACATTCACCAGCTACTGGATGCACTG |
| | | | GGTGAAGCAGAGGCATGGACAAGGCCTTGAGTGG |
| | | | ATTGGAAATATTTATCCTGGTAGTGGTAGTACTA |
| | | | ACTACGATGAGAAGTTCAAGAGCAAGGGCACACT |
| | | | GACTGTAGACACATCCTCCAGCACAGCCTACATG |
| | | | CACCTCGGCAGCCTGACATCTGAGGACTCTGCGG |
| | | | TCTATTACTGTACAATGCGCTACGGTGGTTACTA |
| | | | CGGGTACTACTTTGACTACTGGGGCCAAGGCACC |
| | | | ACTCTCACAGTCTCCTCAGCCAAAACAACAGCCC |
| | | | CATCGGTCTATCCACTGGCCCCTGTGTGTGGAGA |
| | | | TACAACTGGCTCCTCGGTGACTCTAGGATGCCTG |
| | | | GTCAAGGGTTATTTCCCTGAGCCAGTGACCTTGA |
| | | | CCTGGAACTCTGGATCCCTGTCCAGTGGTGTGCA |
| | | | CACCTTCCCAGCTGTCCTGCAGTCTGACCTCTAC |
| | | | ACCCTCAGCAGCTCAGTGACTGTAACCTCGAGCA |
| | | | CCTGGCCCAGCCAGTCCATCACCTGCAATGTGGC |
| | | | CCACCCGGCAAGCAGCACCAAGGTGGACAAGAAA |
| | | | ATTGAGCCCAGAGGGCCCACAATCAAGCCCTGTC |
| | | | CTCCATGCAAATGCCCAGCACCTGAACTCCTGGG |
| | | | GGGACCGTCAGTCTTCCTCTTCCCCCCAAAACCC |
| | | | AAGGACACCCTCATGATCTCCCGGACCCCTGAGG |
| | | | TCACATGCGTGGTGGTGGCAGTGAGCCACGAAGA |
| | | | CCCTGAGGTCAAGTTCAACTGGTACGTGGACGGC |
| | | | GTGGAGGTGCATAATGCCAAGACAAAGCCGCGGG |
| | | | AGGAGCAGTACGCAAGCACGTACCGTGTGGTCAG |
| | | | CGTCCTCACCGTCCTGCACCAGGACTGGCTGAAT |
| | | | GGCAAGGAGTACAAGTGCAAGGTCTCCAACAAAG |
| | | | CCCTCCCAGCCCCCATCGAGAAAACCATCTCCAA |
| | | | AGCCAAAGGGCAGCCCCGAGAACCACAGGTGTAC |
| | | | ACCCTGCCCCCATCCCGGGATGAGCTGACCAAGA |
| | | | ACCAGGTCAGCCTGACCTGCCTGGTCAAAGGCTT |
| | | | CTATCCCAGCGACATCGCCGTGGAGTGGGAGAGC |
| | | | AATGGGCAGCCGGAGAACAACTACAAGACCACGC |
| | | | CTCCCGTGCTGGACTCCGACGGCTCCTTCTTCCT |
| | | | CTACAGCAAGCTCACCGTGGACAAGAGCAGGTGG |
| | | | CAGCAGGGGAACGTCTTCTCATGCTCCGTGATGC |
| | | | ATGAGGCTCTGCACAACCACTACACGCAGAAGAG |
| | | | CCTCTCCCTGTCTCCGGGTAAAGAGGGCAGAGGC |
| | | | AGCCTGCTGACCTGCGGCGACGTGGAGGAGAACC |
| | | | CCGGCCCCATGGTACCGTGCACGCTGCTCCTGCT |
| | | | GTTGGCGGCCGCCCTGGCTCCGACTCAGACCCGC |
| | | | GCGGAAATTGTGCTCACCCAGTCTCCAACCACCA |
| | | | TGGCTGCATCTCCCGGGGAGAAGATCACTATCAC |
| | | | CTGCAGTACCACCTCAAGTATAAGTTCCAATTAC |
| | | | TTGCATTGGTATCAGCAGAAGCCAGGATTCTCCC |
| | | | CTAAACTCTTGATTTATAGGACATCCAATCTGGC |
| | | | TTCTGGAGTCCCAGCTCGCTTCAGTGGCAGTGGG |
| | | | TCTGGGACCTCTTACTCTCTCACAATTGGCACCA |
| | | | TGGAGGCTGAAGATGTTGCCACTTACTACTGCCA |
| | | | GCAGGGTAGTAGTATACCGCTCACGTTCGGTGCT |
| | | | GGGACCAAGCTGGAGCTGAAACGGGCTGATGCTG |
| | | | CACCAACTGTATCCATCTTCCCACCATCCAGTGA |
| | | | GCAGTTAACATCTGGAGGTGCCTCAGTCGTGTGC |
| | | | TTCTTGAACAACTTCTACCCCAAAGACATCAATG |
| | | | TCAAGTGGAAGATTGATGGCAGTGAACGACAAAA |
| | | | TGGCGTCCTGAACAGTTGGACTGATCAGGACAGC |
| | | | AAAGACAGCACCTACAGCATGAGCAGCACCCTCA |
| | | | CGTTGACCAAGGACGAGTATGAACGACATAACAG |
| | | | CTATACCTGTGAGGCCACTCACAAGACATCAACT |
| | | | TCACCCATTGTCAAGAGCTTCAACAGGAATGAGT |
| | | | GT |
| 14 | | Signal peptide | MVPCTLLLLLAAALAPTQTRA |
| 15 | HC | V_{H} | QVQLQQPGSELVRPGASVKLSCKASGYTFTSYWM |
| | | | HWVKQRHGQGLEWIGNIYPGSGSTNYDEKFKSKG |
| | | | TLTVDTSSSTAYMHLGSLTSEDSAVYYCTMRYGG |
| | | | YYGYYFDYWGQGTTLTVSSAKTTAPSVYPLAPVC |
| | | | GDTTGSSVTLGCLVKGYFPEPVTLTWNSGSLSSG |
| | | | VHTFPAVLQSDLYTLSSSVTVTSSTWPSQSITCN |
| | | | VAHPASSTKVDKKIEPRGPTIKPCPPCKCPAPEL |
| | | | LGGPSVFLFPPKPKDTLMISRTPEVTCVVVDVSH |
| | | | EDPEVKFNWYVDGVEVHNAKTKPREEQYNSTYRV |
| | | | VSVLTVLHQDWLNGKEYKCKVSNKALPAPIEKTI |
| | | | SKAKGQPREPQVYTLPPSRDELTKNQVSLTCLVK |
| | | | GFYPSDIAVEWESNGQPENNYKTTPPVLDSDGSF |
| | | | FLYSKLTVDKSRWQQGNVFSCSVMHEALHNHYTQ |
| | | | KSLSLSPGK |

While the foregoing written description of the invention enables one of ordinary skill to make and use what is considered presently to be the best mode thereof, those of ordinary skill will understand and appreciate the existence of variations, combinations, and equivalents of the specific embodiments, methods, and examples herein.

We claim:

1. An antibody or antigen-binding fragment thereof which binds to CD16a, the antibody or antigen-binding fragment thereof comprising a heavy chain variable region and a light chain variable region;
wherein each of the heavy chain and the light chain variable regions comprises a CDR1, CDR2, and CDR3; and wherein:
(g) the sequence of CDR1H comprises sequence GYTFTSYW (SEQ ID NO:1);
(h) the sequence of CDR2H comprises sequence IYPGSGST (SEQ ID NO:2);
(i) the sequence of CDR3H comprises sequence TMRYGGYYGYYFDY (SEQ ID NO:3);
(j) the sequence of CDR1L comprises sequence SSISSNY (SEQ ID NO:4);
(k) the sequence of CDR2L comprises sequence RTS; and
(l) the sequence of CDR3L comprises sequence QQGSSIPLT (SEQ ID NO:5).

2. The antibody or antigen-binding fragment thereof according to claim 1, wherein:
(a) the sequence of the heavy chain variable region comprises a sequence that is at least 90% identical to SEQ ID NO:6; and
(b) the sequence of the light chain variable region comprises a sequence that is at least 90% identical to SEQ ID NO:8.

3. The antibody or antigen-binding fragment thereof according to claim 2, wherein:
(a) the sequence of the heavy chain variable region comprises a sequence that is at least 95% identical to SEQ ID NO:6; and
(b) the sequence of the light chain variable region comprises a sequence that is at least 95% identical to SEQ ID NO:8.

4. The antibody or antigen-binding fragment thereof according to claim 3, wherein:
(a) the sequence of the heavy chain variable region comprises SEQ ID NO:6; and
(b) the sequence of the light chain variable region comprises SEQ ID NO:8.

5. The antibody or antigen-binding fragment thereof according to any one of the preceding claims, wherein the antibody or antigen-binding fragment thereof is a chimeric antibody, a CDR-grafted antibody, or a humanized antibody or antigen-binding fragment thereof.

6. The antibody or antigen-binding fragment thereof according to any one of the preceding claims, wherein the antibody or antigen-binding fragment thereof is a multispecific or a bispecific antibody or antigen-binding fragment thereof.

7. The antibody or antigen-binding fragment thereof according to any one of the preceding claims, wherein the antibody or antigen-binding fragment thereof is an scFv, Fv, Fab′, Fab, F(ab′)₂, or diabody.

8. The antibody or antigen-binding fragment thereof according to any one of the preceding claims, wherein the antibody or antigen-binding fragment thereof has isotype IgG1.

9. The antibody or antigen-binding fragment thereof according to any one of the preceding claims, wherein the antibody or antigen-binding fragment thereof contains D265A N297A (Kabat EU index numbering) substitutions in the constant region of the heavy chain.

10. The antibody or antigen-binding fragment thereof according to any one of the preceding claims, wherein the antibody or antigen-binding fragment thereof is deglycosylated.

11. The antibody or antigen-binding fragment thereof according to any one of the preceding claims, wherein the antibody or antigen-binding fragment thereof is conjugated to one or more of a cytotoxin, a fluorescent label and an imaging agent.

12. An antibody or antigen-binding fragment thereof that binds to the same epitope on human CD16a as the antibody or antigen-binding fragment thereof according to any one of the preceding claims.

13. An isolated nucleic acid or pair of nucleic acids encoding the antibody or antigen-binding fragment thereof according to any one of the preceding claims.

14. A vector or pair of vectors comprising the isolated nucleic acid or pair of nucleic acids according to claim 13.

15. An isolated cell comprising the vector according to claim 14.

16. An isolated cell expressing the antibody or antigen-binding fragment thereof according to any one of claims 1-12.

17. A pharmaceutical composition comprising the antibody or antigen-binding fragment thereof according to any one of claims 1-12 and a pharmaceutically acceptable excipient.

18. A method of reducing CD16a shedding, the method comprising contacting a cell expressing CD16a with the anti-CD16 antibody or antigen-binding fragment thereof according to any one of claims 1-12.

19. The method according to claim 18, wherein the cell expressing CD16a is a NK cell.

20. The method according to claim 18, wherein the cell expressing CD16a is a macrophage.

21. A method of reducing CD16a shedding in a subject in need thereof, the method comprising administering to the subject the antibody or antigen-binding fragment thereof according to any one of claims 1-12.

22. A method of increasing NK cell-driven immunity, the method comprising administering to a subject in need thereof the antibody or antigen-binding fragment thereof according to any one of claims 1-12.

23. A method of increasing NK cell effector function towards cancer cells, the method comprising contacting an NK cell expressing CD16a with the antibody or antigen-binding fragment thereof according to any one of claims 1-12.

24. A method of increasing NK cell effector function towards cancer cells, the method comprising administering to a subject in need thereof the antibody or antigen-binding fragment thereof according to any one of claims 1-12.

25. A method of increasing NK cell-mediated killing of cancer cells, the method comprising contacting an NK cell expressing CD16a with the antibody or antigen-binding fragment thereof according to any one of claims 1-12.

26. A method of increasing NK cell-mediated killing of cancer cells, the method comprising administering to a subject in need thereof the antibody or antigen-binding fragment thereof according to any one of claims 1-12.

27. A method of increasing NK cell-mediated antibody-dependent cellular cytotoxicity (ADCC) towards cancer cells, the method comprising contacting an NK cell expressing CD16a with the antibody or antigen-binding fragment thereof according to any one of claims 1-12.

28. A method of increasing NK cell-mediated ADCC in a subject in need thereof, the method comprising administering to a subject in need thereof the antibody or antigen-binding fragment thereof according to any one of claims 1-12.

29. A method of treating cancer in a subject in need thereof, the method comprising administering to the subject the antibody or antigen-binding fragment thereof according of any one of claims 1-12.

30. The method according to claim 29, wherein the cancer is EGFR+ non-small cell lung cancer (NSCLC), Epidermoid carcinoma, EGFR+ colorectal carcinoma (CRC), Head and neck cancer, B-cell lymphoma, HER2+ breast cancer, Gastrointestinal cancers, PD-L1+ melanoma, Merkel cell and urothelial carcinomas, Squamous cell carcinoma of the lungs, Multiple myeloma, Neuroblastoma, acute or chronic myeloid leukemia, or myeloproliferative neoplasm.

31. A method of reducing tumor growth in a subject in need thereof, the method comprising administering to the subject the antibody or antigen-binding fragment thereof according to any one of claims 1-12.

32. A method of reducing tumor metastasis in a subject in need thereof, the method comprising administering to the subject the antibody or antigen-binding fragment thereof according to any one of claims 1-12.

33. A method of reducing tumor-associated fibrosis in a subject in need thereof, the method comprising administering to the subject the antibody or antigen-binding fragment thereof according to any one of claims 1-12.

34. A method of reducing cancer stemness in a subject in need thereof, the method comprising administering to the subject the antibody or antigen-binding fragment thereof according to any one of claims 1-12.

35. The method according to any one of claims 23, 25, or 27, the method further comprising contacting the cancer cells with a second antibody or antigen-binding fragment thereof, wherein the second antibody or antigen-binding fragment thereof binds a cancer antigen presented by the cancer cell.

36. The method according to any one of claims 21, 22, 24, 26, 28-34, the method further comprising administering to the subject a second antibody or antigen-binding fragment thereof, wherein the second antibody or antigen-binding fragment thereof binds a cancer antigen.

37. The method according to claim 35 or 36, wherein the cancer antigen is selected from the group consisting of CD19, CD30, CD123, CD47, CD33, CD133, BCMA, TEM8, EpCAM, ROR1, Folate Receptor, CD70, MAGE-1, MAGE-2, MAGE-3, MAGE A-10, MAGE-C2, MAGE-A12, CEA, tyrosinase, midkin, BAGE, CASP-8, β-catenin, CA-125, CDK-1, ESO-1, gp75, gp100, MART-1, MUC-1, MUM-1, p53, PAP, PSA, PSMA, ras, trp-1, TRP-1, TRP-2, IL13Ralpha, IL13Ralpha2, AIM-2, AIM-3, NY-ESO-1, C9orf112, SART1, SART2, SART3, BRAP, RTN4, GLEA2, TNKS2, KIAA0376, ING4, HSPH1, C13orf24, RBPSUH, C6orf153, NKTR, NSEP1, U2AF1L, CYNL2, TPR GOLGA, BMI1, COX-2, EGFRvIII, EZH2, LICAM, Livin, Livinβ, MRP-3, Nestin, OLIG2, ART1, ART4, B-cycline, Gli1, Cav-1, Cathepsin B, CD74, E-Cadherin, EphA2/Eck, Fra-1/Fosl 1, GAGE-1, Ganglioside, GnT-V, β1, 6-N, Ki67, Ku70/80, PROX1, PSCA, SOX10, SOX11, Survivin, βhCG, WT1, mesothelin, melan-A, NY-BR-1, NY-CO-58, MN (gp250), telomerase, SSX-2, PRAME, PLK1, VEGF-A, VEGFR2, and Tie-2.

38. The method according to claim 35 or 36, wherein the cancer antigen is selected from the group consisting of epidermal growth factor receptor (EGFR), CD20, HER-2, CD38, programmed death-ligand 1 (PD-L1) and GD2.

39. The method according to claim 35 or 36, wherein the second antibody is selected from the group consisting of cetuximab, rituximab, trastuzumab, avelumab, necitumumab, daratumumab, dinutuximab, zalutumumab, and nimotuzumab.

40. The method according to any one of claims 36-39, wherein the second antibody is administered concurrently with the antibody or antigen-binding fragment thereof according to any one of claims 1-12.

41. The method according to any one of claims 36-39, wherein the second antibody is administered consecutively with the antibody or antigen-binding fragment thereof according to any one of claims 1-12.

42. A kit comprising the antibody or antigen-binding fragment thereof according to any one of claims 1-12 and the second antibody or antigen-binding fragment thereof binds a cancer antigen according to any one of claims 36-39.

43. A method of producing an antibody or antigen-binding fragment thereof, the method comprising:
a. providing a cell expressing the antibody or antigen-binding fragment thereof according to any one of claims 1-12; and
b. isolating the antibody or antigen-binding fragment.
